(19) United States
(12) Patent Application Publication
(10) Pub. No.: US 2024/0000443 A1
(43) Pub. Date: Jan. 4, 2024

(54) NEEDLE AND COLLECTION DEVICE FOR OCULAR BIOPSY AND METHODS OF USE

(71) Applicant: Visionaire Products, Inc., Sewickley, PA (US)

(72) Inventors: Leanne Labriola, Sewickley, PA (US); Rick W. Mental, Villa Park, IL (US); Deana McDonagh, Champaign, IL (US); Thomas Wei, Cary, NC (US); Alex Gorsuch, Sewickley, PA (US)

(73) Assignee: Visionaire Products, Inc., Sewickley, PA (US)

(21) Appl. No.: 18/254,915

(22) PCT Filed: Nov. 30, 2021
(86) PCT No.: PCT/US2021/061277

§ 371 Date: May 30, 2023

Related U.S. Application Data
(60) Provisional application No. 63/119,508, filed on Nov. 30, 2020.

(51) Int. Cl. A61B 10/02

(52) U.S. Cl. CPC A61B 10/0283

(57) ABSTRACT

Provided herein are devices and methods for ocular fluid collection with minimal eye displacement, decreased risk of injury to structures in the eye and reduced ocular fluid sample loss.

### PRIORITY

This application claims priority to U.S. Provisional Application Ser. No. 63/119,508, filed Nov. 30, 2020, which is hereby incorporated by reference in its entirety.

### STATEMENT REGARDING FEDERALLY SPONSORED RESEARCH OR DEVELOPMENT

This invention was made with Government support under NSF SBIR Phase I Grant No. 2051962 awarded by the National Science Foundation. The Government has certain rights in this invention.

### BACKGROUND

Techniques for ocular fluid collection are cumbersome, time consuming, involve loss of ocular fluid for biopsy, and can result in injury to structures of the eye. The high force currently required for needle entry into the eye can create displacement of the eye and can reduce physician control. This increases the risk of injury to the anterior ocular structures. If insufficient fluid volume is collected, then it can compromise the sample or render inaccurate laboratory results. Systems and methods to increase safety, control small fluid volume, and minimize fluid loss are needed.

### SUMMARY

In an embodiment, a device for ocular fluid collection can include: a housing can include a handle and a hub; a first needle having a gauge of 27, 28, 29, 30, 31, 32, 33, or 34; a collection chamber can include a proximal and distal end disposed within the housing and coupled at the proximal end to a distal end of the needle; and a vacuum system within the housing can be configured to pull the ocular fluid through the needle and into the collection chamber, the vacuum system can include: a vacuum chamber; and a compression mechanism connected to the vacuum chamber; and a mechanism to prevent retrograde flow of ocular fluid.

In some embodiments, tubing, a microfluidic system, or a molded polymeric sealed channel connects the collection chamber to the vacuum system. In some embodiments, the device can further include a self-sealing pierceable vacuum septum between the collection chamber and the vacuum chamber. In some embodiments, the needle can include an internal portion between about 14 mm and about 26 mm in length disposed inside the housing and an external portion between about 4 mm and about 6 mm in length disposed outside the housing for insertion into an eye. In some embodiments, the device can further include a mechanism to prevent retrograde flow of ocular fluid that can include a pinch valve, a microfluidic valve, a ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof. In some embodiments, the vacuum system further can include: a vacuum conduit positioned at the distal end of the collection chamber; and a compression mechanism configured to push or pull the vacuum chamber towards or away from the vacuum conduit. In some embodiments, the vacuum conduit can be a second needle. In some embodiments, the distal end of the collection chamber can include a fluid separation membrane configured to prevent collected fluid from exiting the collection chamber or contacting the vacuum chamber. In some embodiments, the device can provide a penetration force between about 0.19 N and about 0.45 N. In some embodiments, the collection chamber can be removable and can be configured to fit into a nucleic acid amplification device, point of care diagnostic, or other diagnostic device. In some embodiments, the device can be configured to collect the ocular fluid in a time period of about 20, 15, 10, 5 seconds or less with a pressure differential between about 50 mmHg to 70 mmHg negative pressure. In some embodiments, the device can further include a visual indicator disposed on an exterior surface of the housing, which indicates when the collection chamber is full. In some embodiments, the device can further include a dolphin nose tip coupled to the housing. In some embodiments, the first needle can include: a proximal and distal end, the proximal end can include a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle; a primary bevel length (L3) between about 0.90 mm and about 1.30 mm; and a lumen length (L2) between about 0.5 mm and 0.9 mm.

In other embodiments, a device for ocular fluid collection can include: a housing can include a handle and a hub; a needle can include a proximal and distal end, the needle can include: a gauge of 27, 28, 29, 30, 31, 32, 33, or 34; the proximal end can include a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle, a primary bevel length (L3) between about 0.90 mm and about 1.30 mm, and a lumen length (L2) between about 0.5 mm and 0.9 mm; a collection chamber within the housing and coupled to the distal end of the needle; and a vacuum system within the housing configured to pull the ocular fluid through the needle and into the collection chamber.

In some embodiments, the needle can include an internal portion between about 14 mm and about 26 mm in length disposed inside the housing and an external portion between about 4 mm and about 6 mm in length disposed outside the housing. In some embodiments, the needle can include a lubricant coating, a hydrophilic polymer coating, an acrylic hydrogel polymer coating, a medical grade silicone lubricant, a crosslinked silicone lubricant, a vapor deposited polymer, a parylene coating, polydimethylsiloxane liquid coating, a dispersion containing 50 percent active silicone mixed in aliphatic and isopropanol solvents, a low residual coating, or combinations thereof. In some embodiments, the device can provide a penetration force between about 0.19 N and about 0.45 N. In some embodiments, the needle can include a total length between about 18.2 mm and about 19.7 mm and a wall thickness of about 0.03 mm to about 0.07 mm. In some embodiments, the collection chamber can be removable and can be configured to fit into a nucleic acid amplification device, point of care diagnostic, or other diagnostic device. In some embodiments, the collection chamber can hold a volume of about 0.05 0.1, 0.15, 0.20, 0.25, 0.30, or 0.35 mL. In some embodiments, the device can further include a dolphin nose tip coupled to the housing. In some embodiments, the device can further include a tube, microfluidic system, or molded polymeric sealed channel connecting the collection chamber to the vacuum system. In some embodiments, the device can further include a valve configured to prevent retrograde flow of the ocular fluid comprising a pinch valve, ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof. In some embodiments, the vacuum system can include a vacuum chamber, a decompression handle, a vacuum conduit, vacuum septum, or combinations thereof. In some embodiments, the vacuum system can be configured to collect the ocular fluid in a time period of about 20, 15, 10, 5 seconds or less with a pressure differential between about 50 mmHg to 70 mmHg negative pressure. In some embodiments, the device can further include a visual indicator disposed on an exterior surface of the housing, which indicates when the collection chamber is full.

In still another embodiment, a device for ocular fluid collection is provided. The device can include: a housing can include a hub and a handle; a device can include a deformable polymeric bulb configured such that mechanical compression of the bulb establishes a pressure differential disposed within a handle portion of the housing when the mechanical compression is released; and a 27, 28, 29, 30, 31, 32, 33, or 34 gauge needle in fluid communication with the a device comprising a deformable polymeric bulb.

In some embodiments, the deformable polymeric bulb can be a transfer pipette comprising a tip portion, a neck portion, and a bulb portion. In some embodiments, the needle can provide a penetration force between about 0.19 N and about 0.45 N. In some embodiments, the ocular fluid can be collected into a tip portion, a neck portion, or a bulb portion of the transfer pipette. In some embodiments, the device can further comprise a ratcheting system that comprises a user operable bulb compression lever (150), a cocking knob (146), and a lock-out pin (148). In some embodiments, the device can further include a retrograde-flow prevention system configured to prevent ocular fluid from being returned to the eye by incorporation of a self-activated lock pin (148) that prevents the user from compressing the bulb without re-cocking the device. In some embodiments, the device can further include a dolphin nose tip coupled to the hub. In some embodiments, the needle can include: a proximal and distal end, the proximal end can include a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about with respect to a transverse axis of the needle; a primary bevel length (L3) between about 0.90 mm and about 1.30 mm; and a lumen length (L2) between about 0.5 mm and 0.9 mm.

In an embodiment, a method for collecting ocular fluid can include: using the devices described herein by inserting a needle into an eye with minimal eye displacement; collecting ocular fluid through the needle into a collection chamber; and removing the needle from the eye.

In some embodiments, the method can further include notifying the user with a signal from an indicator when fluid has been collected into a collection chamber of the device. In some embodiments, the method can further include removing the collection chamber from the device for use in nucleic acid amplification device, point of care diagnostic, or other diagnostic device.

Therefore, provided herein are devices and methods for ocular fluid collection with decreased risk of injury to structures in the eye and reduced ocular fluid sample loss. Devices and methods provide several advantages including, but not limited to: collecting ocular fluid with minimal or no eye rotation during insertion of needle and collection of fluid; collecting ocular fluid with less entry, slide, and exit force to the eye; increased user control and reduced injury to the eye; collecting ocular fluid with minimal or no fluid leakage after ocular fluid extraction; higher volume of fluid collected due to increased safety with extraction; less fluid loss within collecting apparatus; more fluid to send to laboratory for testing; improved results from the ocular fluid procedure leading to improved testing ability, more accurate results and, therefore, better vision outcomes for patients; giving the user more control over the timing of the collection of the fluid from the eye; use by one person and/or one hand, and reducing vision-inhibiting air bubbles.

### BRIEF DESCRIPTION OF THE DRAWINGS

Various objectives, features, and advantages of the disclosed subject matter can be more fully appreciated with reference to the following detailed description of the disclosed subject matter when considered in connection with the following drawings, in which like reference numerals identify like elements.

FIG. 1A shows a schematic diagram of a typical single-bevel needle, showing the bevel angle and the bevel length.

FIG. 1B shows three bevel geometry of a needle according to some embodiments.

FIG. 1C shows three bevel geometry of a needle according to some embodiments.

FIG. 1D shows three bevel geometry of a needle according to some embodiments.

FIG. 1E shows needle radii geometry according to some embodiments.

FIG. 1F shows geometry of a second needle according to some embodiments.

FIG. 1G shows three bevel geometry of a needle according to some embodiments.

FIG. 2A shows a device with the handle portion shown in phantom exposing a hub assembly and vacuum system according to some embodiments.

FIG. 2B shows a hub assembly and vacuum system according to some embodiments.

FIG. 3A shows a perspective view of a including a transfer pipette based vacuum system according to some embodiments.

FIG. 3B shows transfer pipette and air-filled bulb according to some embodiments.

FIG. 4A shows a device with the handle portion shown in phantom exposing a hub assembly and vacuum system according to some embodiments.

FIG. 4B shows a device with the handle portion shown in phantom exposing a hub assembly and vacuum system with a second needle also visible according to some embodiments.

FIG. 5A-5D shows a device including an air-filled bulb based vacuum system that prevents the user from returning fluid back into the eye once fluid is extracted and a lever that displaces air from within the air-filled bulb, according to some embodiments.

FIG. 6A shows a device with the handle portion shown in phantom exposing a hub assembly and vacuum system according to some embodiments.

FIG. 6B shows a device with the handle portion shown in phantom exposing a hub assembly and vacuum system according to some embodiments.

FIG. 7A shows prior to ocular fluid extraction, a user can engage a vacuum by pulling the decompression handle in some embodiments.

FIG. 7B shows a user inserting a needle of a device into the eye, according to some embodiments.

FIG. 7C shows a user engage a vacuum system by depressing a pinch valve button. The rate of vacuum can be controlled by the pressure applied to the pinch valve button, according to some embodiments.

FIG. 7D shows a user removing a device from the eye. Depressing the pinch valve button outside of the eye will engage the vacuum system and clear the needle for ocular fluid collection of residual ocular fluid, according to some embodiments.

FIG. 8A shows a graph showing a force vs. time produced by the ForceTest software for an individual penetration, where all portions of the penetrative forces are labeled.

FIG. 8B shows a graph comparing needle gauge to peak penetrative force (N) through 0.015″ polyurethane film, showing a trend of higher needle gauges resulting in lower penetrative force.

FIG. 9 shows a box and whisker plot displaying results from the 30-gauge needle experiment on film, where the current standard of care needle from BD with a lower bevel angle was shown to have a lower penetration force than the Narang needle with a higher bevel angle.

FIG. 10 shows a box and whisker plot displaying results from the 30-gauge needle experiment on a bovine eye.

FIG. 11 shows a chart showing force results comparing 3-bevel needle and 5-bevel needle penetration forces, where no statistical significance was found.

FIG. 12A shows a hub with a buoyant sphere inside the collection chamber as an indicator according to some embodiments.

FIG. 12B shows a hub with an LED indicator according to some embodiments.

The drawings are not necessarily to scale, or inclusive of all elements of a system, emphasis instead generally being placed upon illustrating the concepts, structures, and techniques sought to be protected herein.

### DETAILED DESCRIPTION

The details of one or more variations of the subject matter described herein are set forth in the accompanying drawings and the description below. Other features and advantages of the subject matter described herein will be apparent from the description and drawings, and from the claims. The disclosed subject matter is not; however, limited to any particular embodiment disclosed.

Needle

Devices for ocular fluid collection described herein can include one, two, or more needles. One or more of the needles can be disposable. In some embodiments, the needle can be a hypodermic needle, which is a very thin, hollow tube with at least one sharp tip.

A needle can be made of any suitable, biocompatible material. The material can be e.g., a metallic alloy, including, but not limited to, titanium, stainless steels such as 304 stainless steel, 316 stainless steel, ETHALLOY® Needle Alloy, and 302 stainless steel, refractory alloys, nitinol, tantalum, as well as various other materials. Any of the needle alloy compositions can contain some percentage of any one or more of nickel, cobalt, chromium, molybdenum, tungsten, rhenium, niobium, etc. Exemplary needles and methods for manufacturing needles can be found in, e.g., U.S. Pat. Nos. 6,018,860 and 8,821,658.

In some embodiments, a needle can include a coating. The coating can be on the exterior surface of the needle, on the interior surface of the needle, or both. A coating treatment on a surface of the needle can assist the needle glide into the eye. A coating can be e.g., a medical grade silicone lubricant, crosslinked silicone lubricant, or vapor deposited polymer to improve the lubricity of the needle during insertion in the eye. A coating can be e.g., a lubricant coating, a hydrophilic polymer coating, an acrylic hydrogel polymer coating, a Dow Corning 360 coating (a clear, colorless polydimethylsiloxane liquid coating), a Dow Corning MDX4-4159 coating (a dispersion containing 50 percent active silicone mixed in aliphatic and isopropanol solvents), a NuSil MED-4162 coating (one-part, silicone dispersion containing high molecular weight hydroxyl-functional silicone polymer dispersed in xylene), a TriboGlide coating (perfluoropolyether chemistry and gas plasma immobilization to create a lubricating coating that is resistant to migration), a parylene coating, or combinations thereof. A coating can be e.g., a low residual coating such that the coating does not accumulate in a sample. A coating can promote capillary flow of capillary fluid through the needle.

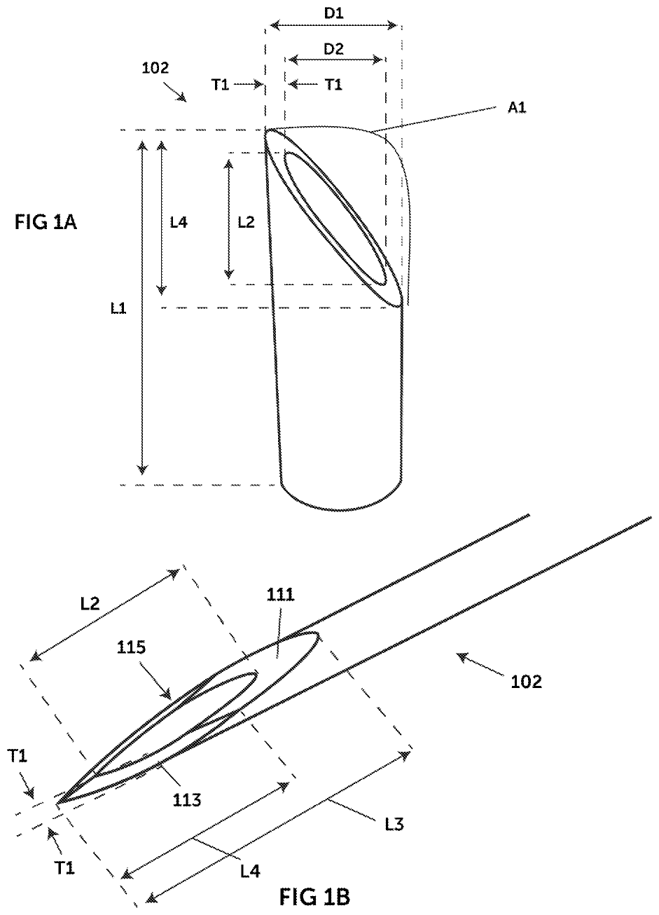

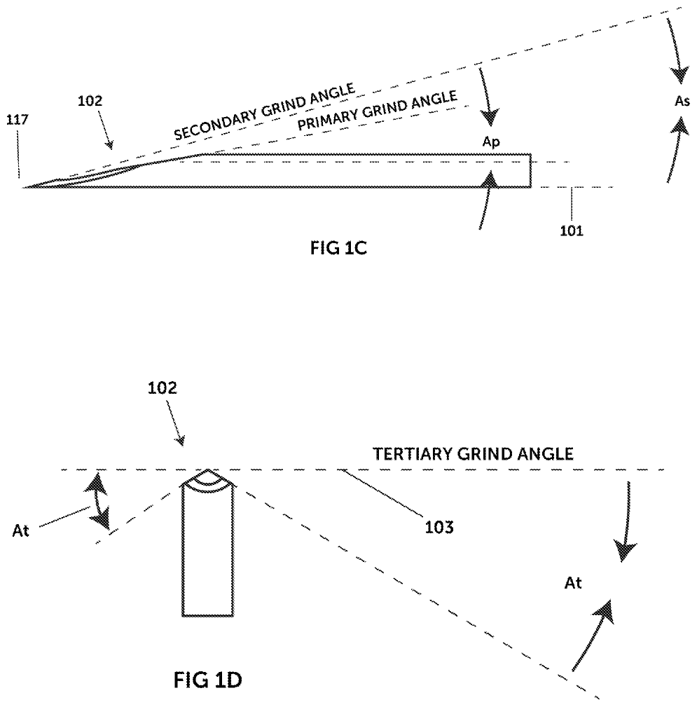

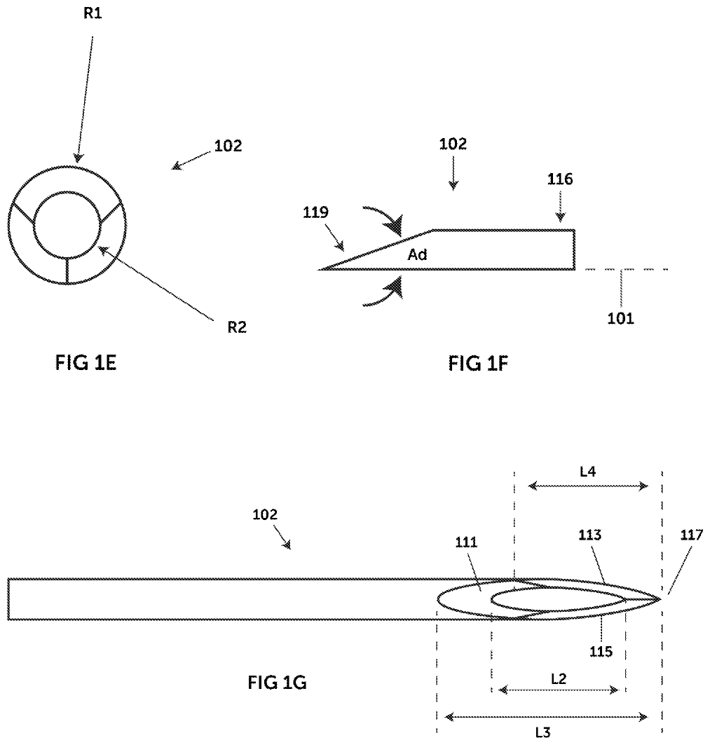

In some embodiments, a needle can include a needle geometry and design optimized for ocular tissue as an ophthalmic tool. A needle can be specialized for entry into the anterior chamber of the eye by passing through the cornea. A needle geometry can include a proximal end and a distal end, a gauge (also referred to as an outer diameter D1), one or more bevel surfaces, and a lumen length (L2). A needle geometry can also include an inner diameter (D2), a wall thickness (T1), and a total length (L1) (see e.g., FIG. 1A).

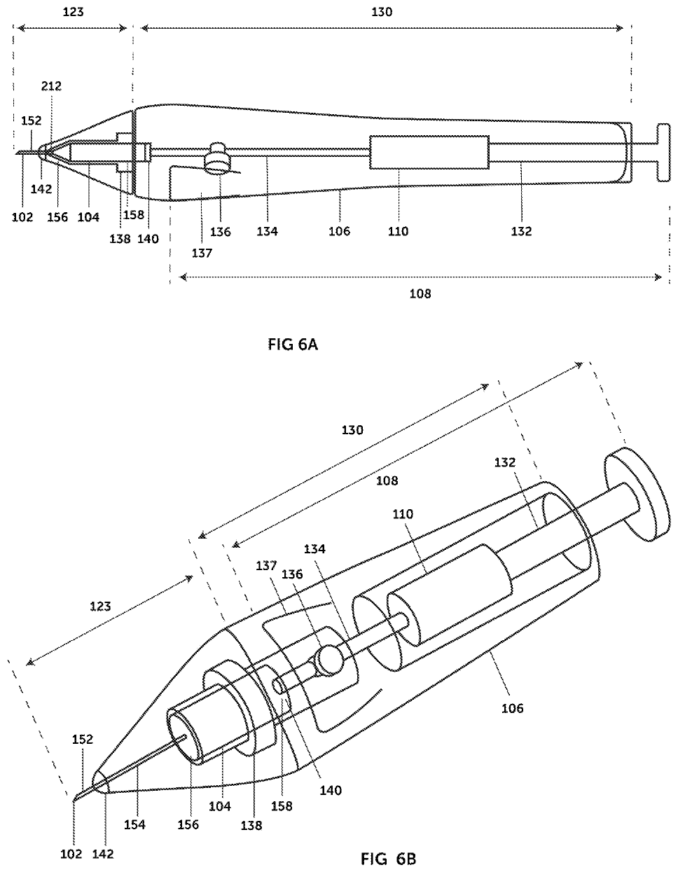

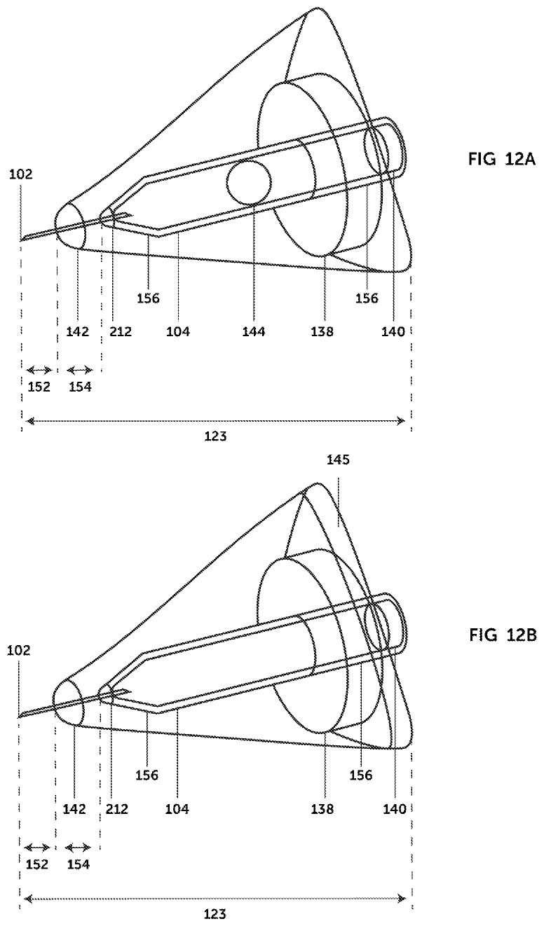

A needle can include a proximal end and a distal end. The proximal end can include one or more bevel surfaces (e.g., 1, 2, 3, 4, 5 or more) and/or one or more bevel lengths. The proximal end of the needle can be inserted into the eye to collect ocular fluid and the distal end of the needle can be directly or indirectly connected to a collection chamber. In some embodiments, a distal end of a needle can connect to a collection chamber by piercing a self-sealing pierceable collection chamber septum (a collection chamber septum) (see e.g., 212 in FIG. 6A and FIG. 12A). In some embodiments, the distal end of a needle can be connected to a check valve, microfluidic system, other apparatus to prevent retrograde flow, or combinations thereof. Retrograde flow is the flow of fluid back into the eye or toward the eye rather than toward the collection chamber. In some embodiments, the distal end of the needle can be connected to a luer attachment, which can be attached to the inner housing of a device and connected to a collection chamber. In some embodiments, a luer connection can be a “quick connect/disconnect” option for connecting a needle for ocular fluid collection to a collection chamber positioned proximal to a vacuum system.

The gauge of a needle can be about 27, about 28, about 29, about 30, about 31, about 32, about 33, or about 34. The gauge can allow for self-sealing of the eye after the needle is removed. A contributor to entrance force is the needle gauge size (i.e., outer needle diameter D1). In some embodiments, the outer diameter of a needle can be between about 0.42 mm and about 0.16 mm. The gauge size, when combined with other needle characteristics, lowers the amount of entrance force, slide force, and exit force for aqueous paracentesis. This allows for devices described herein to reduce the risk of damage to structures in the eye.

In some embodiments, a needle can include one or more beveled surfaces. For example, a proximal end of a needle can include a three-bevel geometry (see e.g., FIG. 1B-1G). A three-bevel geometry can have a primary bevel surface 111 having a primary bevel angle (Ap) (also referred to as a primary grind angle) between about 5° and about 10° (e.g., about 5, 6, 7, 8, 9, or 10°) with respect to a longitudinal axis of the needle, a secondary bevel surface 113 having an angle (As) (also referred to as a secondary grind angle) between about 9° and about 18° (e.g., about 9, 10, 11, 12, 13, 14, 15, 16, 17, or 18°) with respect to a longitudinal axis of the needle, and a tertiary bevel surface 115 having an angle (As) between about 9° and about 18° (e.g., about 9, 10, 11, 12, 13, 14, 15, 16, 17, or 18°) with respect to a longitudinal axis of the needle, the secondary bevel surface and the tertiary bevel surface having one or more of a tertiary bevel angle (At) between about 15° and about 30° (e.g., about 15, 16, 17, 18, 19, 20, 21, 22, 23, 24, 25, 26, 27, 28, 29, or 30°) with respect to a transverse 103 axis of the needle. A tertiary grind angle can be defined as two separate tertiary bevel angles (At) and can be between about 30° and about 60° (e.g., about 30, 32, 34, 36, 38, 40, 42, 44, 46, 48, 50, 52, 54, 56, 58, or 60°) with respect to a transverse axis 103 of a needle.

A bevel geometry of a needle of a device can be orientated so that the bevel geometry of the needle faces upwards toward the operator of the device. The bevel geometry of a needle can be positioned so the bevel is away from patient's iris.

In some embodiments, a needle can have a primary bevel length (L3) between about 0.90 mm and about 1.30 mm (e.g., about 0.90, 0.95, 1.00, 1.05, 1.10, 1.15, 1.20, 1.25, or 1.30 mm).

A needle can have a lumen length (L2) between about 0.5 mm and 0.9 mm (e.g., about 0.5, 0.6, 0.7, 0.8, or 0.9 mm). In some embodiments, the lumen length L2 can be between about 0.07 mm and 2.15 mm (e.g., about 0.07, 0.1, 0.15, 0.2, 0.25, 0.35, 0.4, 0.45, 0.5, 0.55, 0.6, 0.65, 0.7, 0.75, 0.8, 0.85, 0.9, 0.95, 1.0, 1.05, 1.1, 1.15, 1.2, 1.25, 1.3, 1.35, 1.4, 1.45, 1.5, 1.55, 1.6, 1.65, 1.7, 1.75, 1.8, 1.85, 1.9, 1.95, 2.0, 2.05, 2.1, or 2.15 mm) and a bevel angle Al between about 160 and 170° (e.g., about 160, 161, 162, 163, 164, 165, 166, 167, 168, 169 or 170°) (see e.g., FIG. 1A).

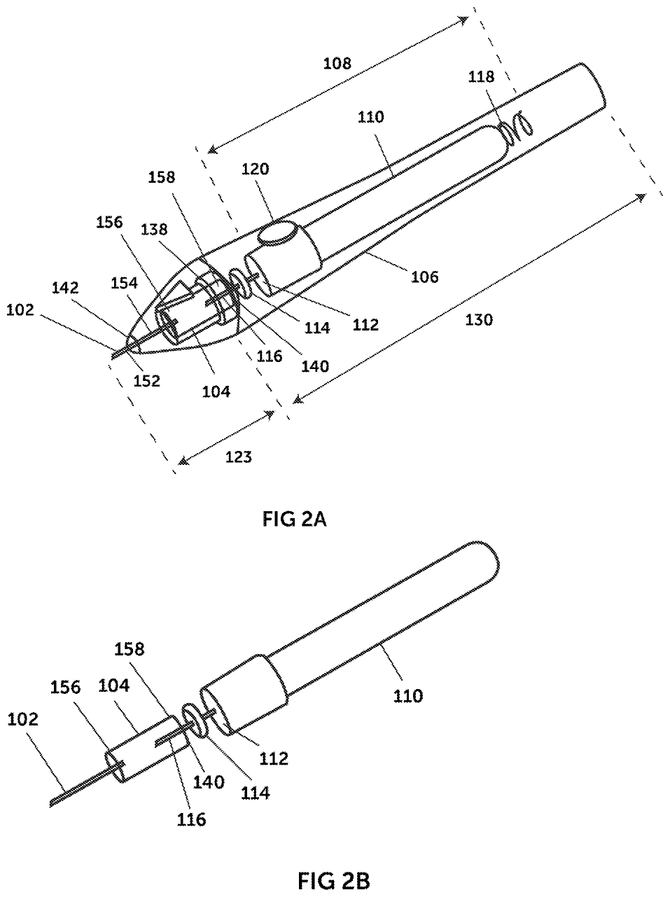

A needle 102 can include a three-bevel geometry including a primary bevel surface 111, a secondary bevel surface 113, and a tertiary bevel surface 115, as shown in FIG. 1B and FIG. 2G. A primary bevel surface 111 can include a primary bevel angle (Ap) of about 3, 4, 5, 6, 7, 8, 9, 10, or 11° with respect to the longitudinal axis 101 of a needle 102 (see e.g., FIGS. 1C-1D). A secondary bevel surface 113 and a tertiary bevel surface 115 can include a secondary bevel angle (As) of about 8, 9, 11, 12, 13, 14, 15, 16, 17, 18, 19, or 20° with respect to the longitudinal axis 101 of a needle 102 (see e.g., FIGS. 1C-1D). A secondary bevel angle (As) of about 18° (e.g., about 8, 9, 10, 11, 12, 13, 14, 15, 16, 17, 18, 19, or 20°) produces a sharper tip 117 at the distal end of the needle 102 for better insertion into the eye (see e.g., FIG. 1C and FIG. 1G). A secondary bevel surface 113 and a tertiary bevel surface 115 can include a tertiary bevel angle (At) of about 14, 15, 16, 17, 18, 19, 20, 21, 22, 23, 24, 26, 27, 28, 29, or about 30° with respect to a transverse axis 103 of a needle 102 (see e.g., FIG. 1D). A secondary bevel surface 113 and the tertiary bevel surface 115 can have one or more of a tertiary bevel angle (At) between about 15° and about 30° (e.g., about 15, 16, 17, 18, 19, 20, 21, 22, 23, 24, 25, 26, 27, 28, 29, or 30°) with respect to a transverse 103 axis of the needle 102. A tertiary grind angle can be defined as two separate tertiary bevel angles (At) and can be between about 30° and about 60° (e.g., about 30, 32, 34, 36, 38, 40, 42, 44, 46, 48, 50, 52, 54, 56, 58, or 60°) with respect to a transverse axis 103 of a needle 102 (see e.g., FIG. 2E). A bevel length and a bevel angle can contribute to an optimal force of entry into an eye.

A needle can include an outside diameter of the needle, which can include a radius of curvature R1 and an inside diameter of the needle can include a radius of curvature R2 (see e.g., FIG. 1E).

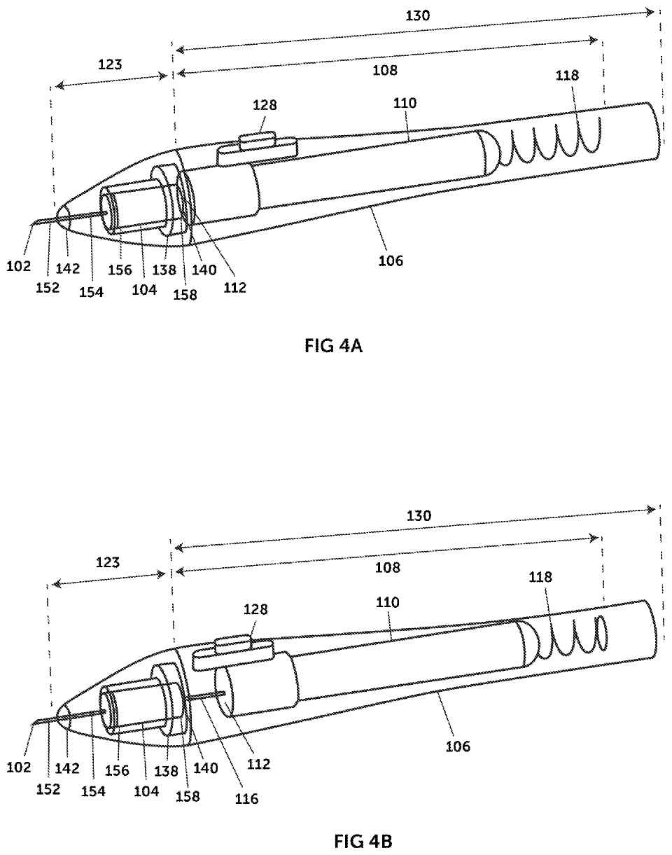

A proximal end of a first needle 102 for fluid collection can include a rear needle point 119 with an angle degree (Ad) of about 25, 26, 27, 28, 19, 30, 31, 32, 33, or about 34° with respect to a longitudinal axis of the needle. An end of second needle 116, which can pierce or operably couple with a vacuum septum 112 or a collection chamber septum 212, can have a needle point with an angle degrees (Ad) of about 26, 27, 28, 19, 30, 31, 32, 33, or about 34° with respect to a longitudinal axis of the needle. See e.g., FIG. 1F, FIG. 2, FIG. 4.

A needle can have a secondary bevel length (L4), which is smaller than L3 (e.g., about 1.25, 1.20, 1.15, 1.10, 1.05, 1.00, 0.95, 0.90, 0.85, 0.80, 0.75, 0.70, 0.65, 0.55, 0.50, 0.45, 0.40, 0.35, 0.30, 0.25, 0.20, 0.15, or 0.10 mm).

In some embodiments, the needle can include an inner diameter (D2). In some embodiments, the inner diameter of the needle can be between about 0.16 mm and about 0.05 mm (e.g., about 0.16, 0.15, 0.14, 0.13, 0.12, 0.11, 0.10, 0.09, 0.08, 0.06, or 0.05 mm).

In some embodiments, a needle can include a wall thickness (T1) between about 0.03 mm and about 0.07 mm (e.g., about 0.03, 0.04, 0.05, 0.06, or 0.07 mm).

A needle can be removable from a device. A needle can be removed from a device for safety and for sanitary purposes. A needle of the device can be disposable.

In some embodiments, needle geometry can be configured to provide an optimal force of entry into the eye. The optimal force of entry can include an entrance force (or penetration force), a slide force, and an exit force. The entrance force is the penetration force. In some embodiments, the entrance force is the force with which the proximal end of the needle enters the eye. The entrance force can be between about 0.1, 0.15, 0.19, 0.20, 0.25, 0.30, 0.35, 0.40, 0.45, 0.50, 0.55, 0.60, 0.65, or A slide force is the force exerted by needle as it moves through the corneal tissue layer. In some embodiments, the slide force can be between about 0.06 and about 0.09 Newton. In some embodiments, the exit force is the force with which the needle is removed from the eye. In some embodiments, the exit force can be between about 0.06 and about 0.09 Newton. In some embodiments the optimal force of entry can be determined based on intraocular pressure as the opposing force preventing the needle from penetrating the eye. Intraocular pressure (IOP) is the fluid pressure inside the eye. Tonometry is a method to determine IOP. In some embodiments, IOP can be an aspect in the evaluation of the needle geometry and the optimal force of entry. In some embodiments, IOP can be measured and can be between about 5, 6, 7, 8, 9, 10, 11, 12, 13, 14, 15, 16, 17, 18, 19, or about 20 mmHg. A reduction in the optimal force of entry prevents, e.g., injury to the eye.

In some embodiments, a needle can include an interior portion 154 and an exterior portion 152. The interior portion 154 of a needle can be contained within a hub or a housing of a device. The exterior portion 152 of a needle can extend outside the hub of the housing and can be exposed to the eye. An interior portion 154 of a needle can be between about 14 mm and about 26 mm (e.g., about 14, 15, 16, 17, 18, 19, 21, 22, 23, 24, 25, or 26 mm) in length. A distal end of a needle can be an interior portion 154 of a needle. An exterior portion 152 of a needle can be between about 4 mm and about 6 mm (e.g., about 4, 4.5, 5, 5.5, or 6 mm) in length. The length of the exterior portion of the needle can ensure good safety and proper penetration force. A proximal end of a needle can be an exterior portion 152 of a needle. An exterior portion 152 of a needle can be the length of a needle extending from a dolphin nose tip, a hub, or a housing. In some embodiments, a rear needle point 119 of a first needle for fluid collection can pierce a collection chamber septum located at a proximal end of a collection chamber.

In some embodiments, a needle can withdraw ocular fluid. A needle can be designed specifically for collection of fluid from the anterior chamber of the eye. The fluid can be e.g., aqueous fluid, ocular fluid, tear film fluid, periocular tissue fluid, or the like. A needle can include a specific geometric configuration of a bevel length and bevel angles that enables optimal force of entry.

In some embodiments, a device can include a second needle, disposed inside the housing of the device between a collection chamber and a vacuum system. A second needle can be affixed to a vacuum conduit. A second needle can be used to pierce a self-sealing pierceable vacuum septum (a vacuum septum) after the first needle has been fully inserted into the eye. The vacuum is activated by a vacuum chamber release button so that the vacuum chamber is pushed towards and onto the second needle, thereby establishing a pressure differential (see e.g., FIG. 2 and FIG. 4). In some embodiments, a second needle can be removed and discarded when a collection chamber is removed from a device.

Check Valve

In some embodiments, a device can include a check valve positioned at a distal end of a needle (for example, between the distal end and the collection chamber) for ocular fluid collection to prevent retrograde flow of fluid. In some embodiments, a vacuum system, microfluidic system, a fluid separation membrane, or combinations thereof can prevent retrograde flow of fluid without the need for a check valve. Thus, in some embodiments, a device does not include a check valve. A check valve has two-port valves, meaning it has two openings in the body, one for fluid to enter and the other for fluid to leave. A check valve can be referred to as a non-return valve, reflux valve, retention valve, foot valve, one-way valve, or the like. A check valve is a valve that normally allows fluid (gas) to flow through it in only one direction. Various types of check valves can be used, such as, but not limited to, a ball check valve, a diaphragm check valve, a swing check valve, a flapper valve, a stop-check valve, a lift-check valve, an in-line check valve, a duckbill valve, a pneumatic non-return valve, or a Tesla check valve. Check valves work automatically and are not controlled by a person or any external control; accordingly, they do not have any valve handle or stem. Check valves have a cracking pressure, which is the minimum differential upstream pressure between inlet and outlet at which the valve will operate. Typically, a check valve can be designed for, and can therefore be specified for, a specific cracking pressure (e.g., 11, 10, 9, 8, 7, 6, 5, 4, 3, 2, 1, or less mmHg). In some embodiments, a check valve can be a Tesla check valve, which includes a valvular conduit, such as a fixed-geometry passive check valve. A Tesla check valve allows a fluid to flow in one direction, without moving parts.

Collection Chamber

A device can include a collection chamber, which can collect ocular fluid. A collection chamber can be a vial, an Eppendorf tube, a microcentrifuge tube, a tube, a hollow container, a container, a chamber, a casing, a tip, neck, or bulb of an air-filled bulb device such as a transfer pipette, or any other suitable container. In some embodiments, a collection chamber can include a cap. A cap can be a snap top, a screw cap, a self-sealing elasticized stopper, or the like. A collection chamber can hold a volume of about 0.05, 0.1, 0.15, 0.20, 0.25, 0.30, 0.35 mL or more. A collection chamber can be removed from a device for further analysis of the ocular fluid. A collection chamber can be configured for point of care use. A collection chamber can be a standard size and can be used directly into a nucleic acid amplification device, point of care diagnostic, or other diagnostic device. A collection chamber can have a proximal end and a distal end.

A collection chamber can be made of any suitable material, e.g., polypropylene, molded plastic, glass, or other suitable materials. A collection chamber can be made of a material that protects the sample from UV light. UV light protective material can prevent denaturation or degradation of a sample contained within a collection chamber. A UV material can be, e.g., polyethylene tubing.

A collection chamber can be disposed within the housing of the device between a needle and a vacuum system.

A collection chamber can be operably connected to a luer lock, luer connection, or luer device at a distal end of the collection chamber. In some embodiments, a luer connection can be a “quick connect/disconnect” option for connecting a needle for ocular fluid collection to a collection chamber positioned proximal to a vacuum system.

The interior portion or a distal end of a needle can be connected to a collection chamber. A collection chamber can have a self-sealing pierceable septum or can be directly connected to a first needle for fluid collection. A collection chamber can be connected to an elastomeric or other suitable tubing (e.g., a metal or molded polymeric sealed channel) or a microfluidic system, which is in turn connected to the needle for fluid collection. Where a collection chamber has a self-sealing pierceable septum the collection chamber can be pierced with a needle at a proximal end of the collection chamber to begin fluid collection and then the collection chamber can be capped at distal end when the fluid collection is complete. In some embodiments, a distal end of a collection chamber can have one cap used during collection and subsequent transit and/or analysis. In some embodiments a proximal or distal end of a collection chamber can have a cap used during collection that contacts fluid and therefore is disposed after collection and a second cap used to seal the distal end of a collection chamber after fluid collection is complete. In some embodiments, a collection chamber can include a self-sealing pierceable collection chamber septum at a proximal end of a collection chamber that is pierced with a needle to begin fluid collection.

In some embodiments, a collection chamber can include a collection chamber septum at a proximal end of the collection chamber. A collection chamber septum is a self-sealing pierceable collection chamber septum that can provide a vacuum seal to a distal end of a needle and function as a liquid seal to prevent leakage of collected ocular fluid after a collection chamber is removed from a device (e.g., for transit to laboratory for analysis or transit within point-of care facility for analysis).

A collection chamber can include a fluid separation membrane (also referred to as a semipermeable membrane and a sealing mechanism) at a distal end of the collection chamber to prevent any collected fluid traveling beyond the collection chamber and into a vacuum chamber. A fluid separation membrane can provide a passive means to prevent fluid that has been collected from the eye into a collection chamber from leaking out of the collection chamber through a distal end of the collection chamber through a pathway that connects a vacuum system to the collection chamber. A fluid separation membrane can have a media that can have a precisely controlled porosity that can enable air to pass freely but can completely block the passage of any liquid material. The fluid separation membrane may be a die cut or otherwise shaped membrane that is precisely fitted into the distal end of a collection chamber. A fluid separation membrane can be positioned so as not to be penetrated by a proximal end of a vacuum conduit that may be pre-installed or inserted in a distal end of a collection chamber just prior to device use. A fluid separation membrane can be a semi-permeable membrane that can also be found within a collection chamber that separates fluid from tissue collected from the eye. A fluid separation membrane can be disposed between a collection chamber and a vacuum conduit to seal the collection chamber after the fluid is disposed in the collection chamber. A needle can direct the fluid from the eye into a collection chamber and avoid fluid loss or retrograde flow. In some embodiments, a needle can be connected to elastomeric or other suitable tubing (e.g., a metal or molded polymeric sealed channel) or a microfluidic system configured with a fluid separation membrane to separate fluid collected.

A collection chamber can be connected to a vacuum conduit. A vacuum conduit is the pathway that connects a vacuum chamber to a collection chamber. A vacuum conduit can include individual components (e.g., a conduit such as a second needle; or tubing and a valve) or molded-in features (e.g., a conduit designed with an integral stopper, control disk, or safety stop) that provide an airtight passageway between a vacuum source (e.g., a vacuum system), and a collection chamber which is connected to a first needle for ocular fluid collection. A vacuum conduit can be installed into a distal end of a collection chamber. A vacuum conduit can be connected to a second needle at a distal end of a collection chamber and the second needle can pierce a vacuum chamber septum thereby connecting a vacuum chamber to the collection chamber and creating an artificial pressure differential along the fluid path. A vacuum conduit can allow for activation of a vacuum system after the needle is inserted into the eye. Alternatively, a rear needle point 119 of a first needle for fluid collection can pierce a vacuum chamber septum.

In some embodiments, a valve can be configured to prevent over deflation of the eye from a vacuum system can be positioned proximal to a collection chamber.

Vacuum System

A device can include a vacuum system. The vacuum system can include a vacuum chamber and a vacuum septum (e.g., a self-sealing pierceable vacuum septum). A vacuum system can include a valve, a compression mechanism, and/or a microfluidics system.

A vacuum chamber can be a tube, a vial, a pipette, a bulb, or any other suitable container, wherein air and other gases are removed, which results in a low-pressure environment within the vacuum chamber. In some embodiments a vacuum chamber is not a perfect vacuum. A vacuum chamber can be manufactured to provide about 10 mmHg or less (e.g., about 10, 9, 8, 7, 6, 5, 4, 3, 2 or 1 mmHg). A vacuum chamber can be disposable. A vacuum chamber can remain free of any contact by a biological sample such as ocular fluid.

An amount of vacuum in a vacuum chamber can be directly proportional to force needed to take fluid from eye, draw fluid out of needle, and deposit fluid into a collection chamber. A vacuum chamber can be modulated to change force with which fluid is withdrawn. A vacuum chamber can clear fluid from the needle of the device and deposit all the fluid in a collection chamber such that no fluid is wasted.

A vacuum system can direct the fluid from the eye into the collection system and avoid fluid loss or retrograde flow. Retrograde flow is the flow of fluid back into the eye or toward the eye rather than toward the collection chamber. A vacuum system can prevent fluid loss within the needle head and prevent the transfer of the fluid out of a collection chamber after collection. In some embodiments, the vacuum system can also prevent air going back into the eye after collection.

A vacuum system can include a vacuum septum (e.g., a self-sealing pierceable vacuum septum). A vacuum septum can be attached to a proximal end of a vacuum chamber. A vacuum septum can be positioned between a collection chamber with a vacuum conduit and a vacuum chamber. A vacuum chamber can be pierced by a sharpened distal end 119 of a first needle for fluid collection or a second needle acting as a vacuum conduit, thereby connecting the vacuum chamber to the collection chamber, and creating an artificial pressure differential along the fluid path. The needle that pierces a vacuum septum can be removed and disposed of.

A vacuum system can include a valve, which can be attached to the vacuum conduit. A valve can be a pinch valve or similar valve coupled to the vacuum chamber or vacuum conduit (e.g., elastomeric or other suitable tubing such as a metal, a molded polymeric sealed channel, or a microfluidic system, see e.g., FIG. 6), which can be configured to control the amount of vacuum force used in the system.

A vacuum system can include a compression mechanism. A compression mechanism can be, e.g., a spring mechanism, a decompression handle mechanism, a ratcheting mechanism, a bulb-based mechanism or other suitable mechanism. A compression mechanism can be called a vacuum chamber release mechanism and can be configured to push or pull the vacuum chamber towards or away from a vacuum conduit, wherein a needle pierces a vacuum septum (e.g., a self-sealing septum) The user-controlled movement of the mechanism can be used to modulate the flow of vacuum system thereby controlling the rate fluid is drawn into a collection chamber.

A spring compression mechanism can include a vacuum chamber and a spring or a spring-loaded mechanism. Initially, a vacuum chamber can be inserted into a distal end a handle of a device and pushed towards a distal end of a handle thereby compressing a spring until a latch (e.g., a retention latch mechanism or slider with a latch) grips a proximal end of the vacuum chamber securing the vacuum chamber in place. Once a proximal end of a needle for ocular fluid collection is inserted into the eye creating a secure and enclosed environment, a vacuum chamber can be activated. A vacuum chamber is activated when a latch release (e.g., a retention latch mechanism or slider with a latch) is activated by the user allowing vacuum chamber to engage with a collection chamber. The spring will decompress pushing a second needle at a distal end of a collection chamber through a vacuum chamber septum; this will create a vacuum from the vacuum chamber, through a vacuum conduit, through a second needle, through a collection chamber, through a needle for ocular fluid collection as the needle is connected to the eye which will allow the fluid from the eye with higher pressure to drain into a device with lower pressure under vacuum, see, e.g., FIG. 2 and FIG. 4). A spring compression mechanism can include a slider (e.g., a slide) and at least one spring operably coupled to a distal end of a vacuum chamber and disposed inside a housing of a device (see e.g., FIG. 4). A slider can move the vacuum chamber forward toward a distal end of a collection chamber of a device using the slider on a handle portion of a housing, thereby decompressing the spring. A slider can be positioned on top of a handle portion of a housing or at any other suitable position. The vacuum chamber (or vacuum septum, for example) can be pierced by a second needle extending from a distal end of a collection chamber. This configuration can give the user more control over the timing of the collection of the fluid from the eye by the needle by allowing intermittent engagement of a vacuum chamber with a fluid collection path (see e.g., FIG. 4). A spring compression mechanism can include a retention latch mechanism 120 and at least one spring operably coupled to a distal end of a vacuum chamber and disposed inside a housing of a device (see e.g., FIG. 2). A retention latch mechanism 120 can include a button, a hook, a barrier or any other suitable components. Initially, a vacuum chamber can compress a spring located in a handle at a distal end of the vacuum chamber and a retention latch mechanism 120 can secure the vacuum chamber this position. Then, a distal end of a collection chamber can be inserted into a proximal end of a hub assembly. A collection chamber can have a vacuum conduit installed into a proximal 156 end of the collection chamber. An assembled needle, hub assembly (including e.g., a collection chamber and a vacuum conduit) can be attached to a handle. Upon a user fully inserting a needle 102 into the eye, the user can press, slide or otherwise engages a retention latch mechanism 120, which decompresses a spring and pushes a distal end of a vacuum chamber towards a proximal end of a vacuum conduit which then pierces a vacuum chamber septum 112 thereby connecting the vacuum chamber to the collection chamber, creating an artificial pressure differential along the fluid path (see e.g., FIG. 2). In other words, a retention latch mechanism can allow a vacuum system to be turned on or turned off by engaging the spring mechanism. A collection chamber can include a fluid separation membrane at the distal end to prevent any collected fluid traveling beyond the collection chamber and into the vacuum chamber (see e.g., FIG. 2 and FIG. 4). A device with a spring compression mechanism can prevent the risks of contamination and vision-inhibiting air bubbles in the eye (see e.g., FIG. 2 and FIG. 4).

A decompression handle mechanism can include a vacuum chamber and a decompression handle. A decompression handle can be operably coupled to a vacuum chamber by means of an air-tight seal and when pulled and extended in a locked position can evacuate gas from the vacuum chamber thereby establishing a vacuum state in the vacuum chamber. A device with a decompression handle mechanism can further include elastomeric or other suitable tubing (e.g., a metal or molded polymeric sealed channel or microfluidic system) disposed between a vacuum chamber operably coupled to a decompression handle and a collection chamber, which can be coupled to a needle for ocular fluid collection (see e.g., FIG. 6). The elastomeric or other suitable tubing (e.g., a metal or molded polymeric sealed channel or microfluidic system) can be considered a vacuum conduit. Initially, a user can pull back the decompression handle, which applies a vacuum force through the elastomeric or other suitable tubing (e.g., a metal or molded polymeric sealed channel or microfluidic system), the collection chamber, and the needle to withdraw fluid from the eye. In some embodiments, a decompression handle can be pulled back from the housing in preparation of using the device. A decompression handle can be pulled out while using the device (e.g., during fluid collection). A decompression handle can be configured such that when the decompression handle is touching a housing of a device, that can indicate the pressure in a vacuum chamber no longer functions as a vacuum.

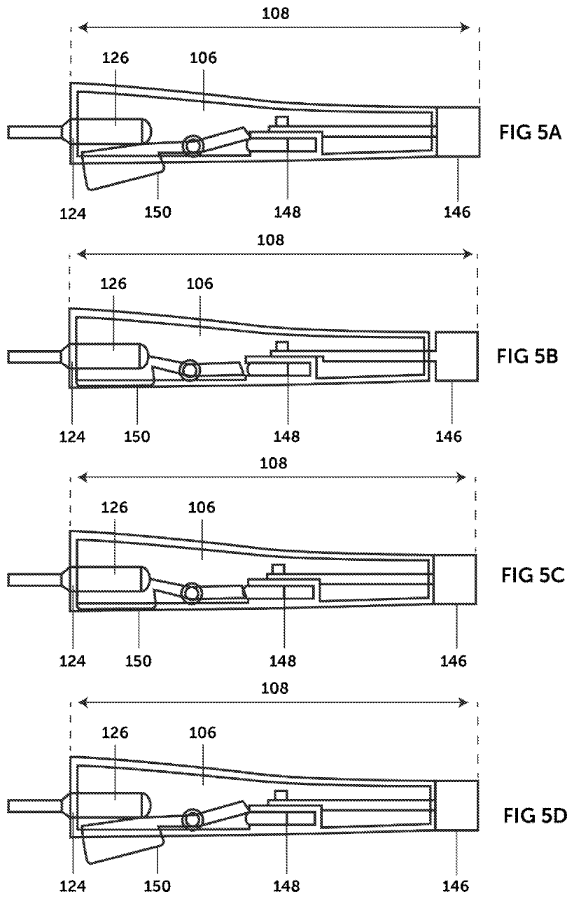

A ratcheting compression mechanism (i.e., ratcheting system) can include a vacuum chamber, a lever, a cocking knob, and a lock-out pin. A ratcheting compression mechanism can compress (deform) a deformable air-filled bulb of a transfer pipette to establish a pressure differential using a user operable bulb compression lever (150) that cannot be depressed until the cocking knob (146) is pulled outward from a handle, pulling with it a lock-out pin (148) (see e.g., FIG. 5). Air-filled bulb compression can be maintained by the user continuing to press on an exposed portion of a lever (150), the user slowly releases the lever (150) only after fully inserting a needle into the eye, when an air-filled bulb has fully decompressed a spring (not shown) pushes a lock pin (148) forward and under the end of the lever thereby preventing the user from compressing the bulb without re-cocking a device (see e.g., FIG. 5). A device can include a retrograde-flow prevention system configured to prevent ocular fluid from being returned to the eye by incorporation of a self-activated lock pin (148) that prevent the user from compressing the bulb without re-cocking the device (see e.g., FIG. 5).

An air-bulb based compression mechanism can comprise, for example, a transfer pipette or other suitable device. A user can mechanically compress (deform) a deformable bulb of a transfer pipette to establish a pressure differential when the mechanical compression is released and the bulb returns to its original shape and volume. Prior to inserting the needle into the eye, a bulb can be compressed by the user pressing, sliding, cocking, collapsing or otherwise engaging a lever-type mechanism that compresses the bulb. Compression of the bulb can be released after the eye has been punctured and a needle is fully inserted into, e.g., the anterior chamber, the negative pressure created by the expanding bulb combined with the pressure within the eye allows for quick fluid extraction into a collection chamber. Upon completion a collection chamber component can be removed, properly packaged and shipped to a lab for analysis by a nucleic acid amplification device (e.g., a PCR device), point of care diagnostic, or other diagnostic procedure. A transfer pipette can include a pipette with a pierceable collection chamber septum located at a proximal end of the pipette section to prevent retrograde fluid flow once collected. In some embodiments, collected fluid can be free to flow into both the pipette neck and bulb areas of a transfer pipette; when ready for analysis the transfer pipette can be held with the pipette neck section point upwards and a tip of the pipette neck including a collection chamber septum can be cut off; the remaining portion of the transfer pipette can then be inverted so the collected fluid could be discharged into the appropriate container for PCR or other analysis.

A device can include a microfluidic system to maintain fluid inside the collection chamber, prevent fluid retention with the device, reduce fluid loss from the procedure, and prevent retrograde flow. Retrograde flow is the flow of fluid back into the eye or toward the eye rather than toward the collection chamber. Microfluidics systems are miniaturized collection devices that offer fast fluid collection speeds with high efficiencies, which can be used to obtain small sample or biopsy sizes. A microfluid system controls flow, direction and collection of fluid to minimize fluid loss and can be used to separate fluid into separate compartments of a device for collection or separate fluid from ocular tissue or other contaminates collected. A microfluidics system can be e.g., flow-based channel microfluidics, electric-based digital microfluidics (DMF), or other suitable systems.

In some embodiments, the microfluidic system can include components such as channels, reservoirs, vacuum sources, valves, collection chambers, filters, fluidic interconnects, diffusers, and other microfluidic components. These microfluidic components typically have dimensions between a few micrometers and a few hundreds of micrometers. The small dimensions of the components of the microfluidic system can enable efficient management of fluid movement and minimize the physical size, response time, and waste (i.e., fluid loss) within the system. A configuration of the microfluidic system itself can help direct fluid into a single collection chamber or to two different collection chambers. A microfluidic system can withdraw fluid from a dead space of a needle head. The dead space of a needle is the area of the needle head where fluid can remain against the walls of the needle head which prevents it from being collected in a collection chamber and able to be sent to a laboratory for testing. A microfluidic system also prevents retention of fluid in a dead space of a syringe. Internal components of the microfluidics system can contain low residual coatings.

A microfluidic system can be designed for small fluid volumes and for ocular fluid. The microfluidic system can include a microfluidic chamber. The microfluidic chamber size can be optimized to allow about 0.05, 0.1, 0.15, 0.20, 0.25, 0.30, 0.35 mL or more fluid to be collected. Other fluid collection systems are limited by the retention of fluid in the device even after transfer of the fluid for testing. On average 0.05 mL of fluid can remain in prior art system syringes.

In some embodiments a microfluidic system can prevent fluid loss within the needle head the transfer of the fluid after collection. A microfluidic system can prevent air going into the eye after collection. A microfluidic system can connect a needle to a vacuum system.

A microfluidic system can be positioned between a needle and a collection chamber and/or can be pare of a vacuum conduit or a vacuum system.

Valve System

A device can include a valve system, which can include elastomeric or other suitable tubing (e.g., a metal tube, molded polymeric sealed channel), a microfluidic system, a pinch valve, or combinations thereof. In some embodiments, a valve system can be a used to control the vacuum distal to the collection chamber and to control fluid flow anterior to the collection chamber in the system.

A valve system can separate the vacuum system from the collection chamber. In some embodiments, the valve system can be disposed in the housing of the device between the collection chamber and the vacuum chamber. In some embodiments, a valve system can be a vacuum conduit.

A valve system can include elastomeric or other suitable tubing (e.g., a metal tube or molded polymeric sealed channel). The tubing can be, e.g., a compound of a polyethylene terephthalate copolymer and polyethylene naphthalate copolymer, a copolymerized polyester resin containing terephthali acid and isophthalic acid as acid components and ethylene glycol as a diol component and having crystallinity, and the like. In some embodiments, a thin film layer of ceramic may be formed on the surface (inner peripheral surface and/or outer peripheral surface) of an ethylene-polypropylene random copolymer by a plasma chemical vapor deposition (CVD) method. Furthermore, the inner surface of the elastomeric tubing can be made of plastic can be coated with a thin film of silicon oxide containing a carbon atom.

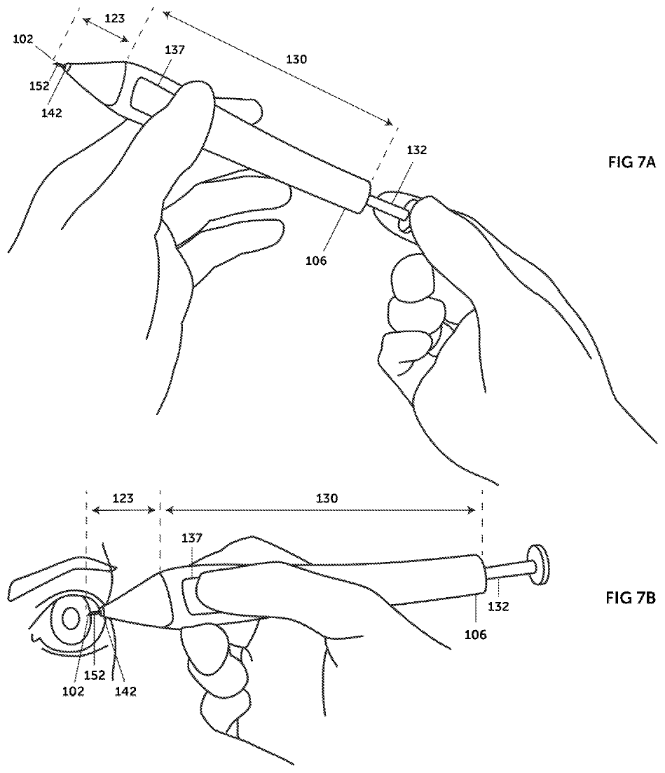

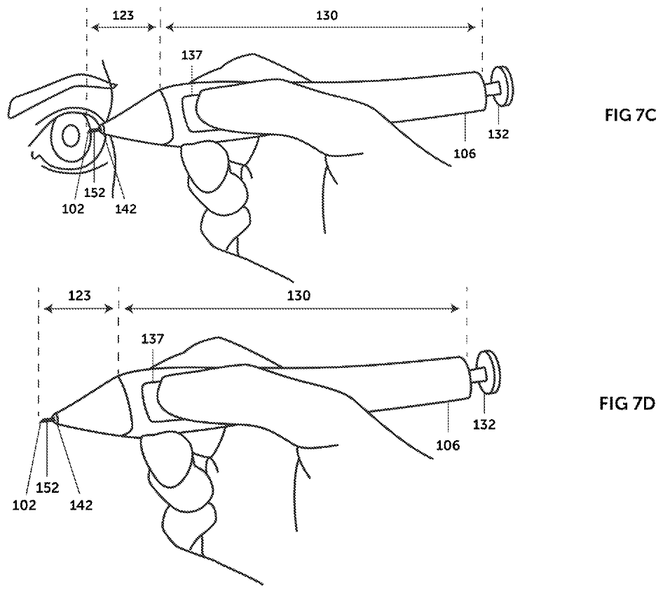

A valve system can include, e.g., a pinch valve. A pinch valve can be disposed inside a housing and can be removably attached to elastomeric or other suitable tubing (e.g., a metal tube or molded polymeric sealed channel). A pinch valve can flatten the elastomeric or other suitable tubing (e.g., a metal or molded polymeric sealed channel) to control the flow of vacuum and can be elevated away from elastomeric or other suitable tubing (e.g., a metal or molded polymeric sealed channel) to induce flow. A pinch valve can be modulated by a user through a pinch valve access button 137 on a handle of a device (see e.g., FIG. 7).

Sealing Mechanism

In some embodiments, a fluid separation membrane can be configured with a sealing mechanism. In some embodiments, before fluid collection, a sealing mechanism can seal an opening of a microfluidic system. In some embodiments, before fluid collection, a sealing mechanism can seal an opening of a microfluidic passageway or chamber within a microfluidics system, when the sealing mechanism is in close contact with an opening of a check valve (at a proximal end of a collection chamber), so that a reduced pressure state inside a needle is maintained. In some embodiments, during fluid collection, a proximal end of a needle can be inserted into an eye of a subject, and then, due to the pressure difference between the pressure inside the eye tissue and the pressure inside a needle and a collection chamber, the collection of fluid from the subject can be performed. In some embodiments, the pressure differential can between about 6, 7, 8, 9, 10 11, 12, 13, 14, 15, 16, 17, 18, 19, 20, 21, 22, 23, 24, or 25 mmHg. Thus, in some embodiments, fluid collection can be performed by a vacuum system. In some embodiments, even after a needle is pulled out of the eye, the pressure of a vacuum system can be used to clear the remaining fluid from a system and force the fluid from a needle and a microfluidic system into a collection chamber. In some embodiments, a sealing mechanism can be elastically deformed to close a hole in a semipermeable membrane from a needle that was connected to the vacuum, so that liquid tightness can be kept in the collection chamber. Therefore, in some embodiments, a collected ocular fluid sample can be prevented from leaking from a distal needle hole that was used to connect a vacuum system to a collection chamber during fluid collection.

In some embodiments, the pressure can be reduced inside the collection chamber, where the sealing mechanism is brought into contact with the microfluidic system, so that airtightness can be kept, therefore, due to the pressure difference between the air pressure inside the chamber and atmospheric pressure, and reduced pressure state inside the collection chamber before fluid collection can be maintained.

Indicator

in some embodiments, a device can include an indicator. An indicator can be positioned on an exterior surface of a housing of a device. An indicator can be a light a signal, or any other visual indication. A portion of the hub containing a collection chamber can be clear or transparent such that a user can see when the collection chamber is full. An indicator can signal when a collection chamber is full. An indicator can be a LED light 145 indication (see e.g., FIG. 12B), a buoy system that signals when the collection chamber is full (see e.g., FIG. 12A), based on an index of refraction system, be based on a color change, an electronic indicator, or any other suitable method. An indicator can use internal sensors to detect when the collection chamber is full. An indicator can detect when the collection chamber is about half full, nearly full, or full. In some embodiments, an indicator 144 can be a small buoyant sphere inside a collection chamber 104 and visible through a hub 123 that can be made of transparent material (see e.g., FIG. 12A).

In some embodiments, when a device is used by an operator, the indicator can be a visual cue that appears once about 0.05, 0.1, 0.15, 0.20, 0.25, 0.30, 0.35 mL or more fluid has been collected into the collection chamber.

Housing

The device can include a housing containing components of the device. A housing can contain the collection chamber, a portion of the needle, a valve system, a microfluidic system, a vacuum system, a vacuum chamber, a check valve, an indicator, a combination thereof or other suitable elements.

A housing can be tubular, triangular, hour-glass shaped, curved, rounded, or any other suitable shape.

A housing can include a handle portion. A handle portion can include ergonomic features. Ergonomic features can include shaping for hand positing and comfort, texture for grip, or other suitable designs. A texture can be rubberized. The housing can be configured so that the device can be for single-operator use. The housing can include one or more buttons, slides, latches, or other features on the outer surface. One or more buttons, slides, latches, or other features can control the vacuum system. A housing of a device can orient a needle direction and bevel direction for safety.

The housing can allow for visualization of ocular fluid collection in a collection chamber.

The housing can allow for orientation of a bevel tip of a needle for proper direction for insertion into the eye to prevent damage.

The housing can be about 40, 45, 50, 55, 60, 65, 70, 75, 80, 85, 90, 95, 100, 105, 110, 115, 120, 125, 130, 135, 140, 145, 150, 155, 160, 165, 170, 175, 180 mm, or more in total length from the proximal to distal end. The handle portion of the housing can be about 40, 45, 50, 55, 60, 65, 70, 75, 80, 85, 90, 95, 100, 105, 110, 115, 120, 125, 130 mm, or more in length. The housing can be about 2, 3, 4, 5, 6, 7, 8, 9, 10, 11, 12, 13, 14, 15, 16, 17, 18, 19, 20 mm or more thick at the widest point.

In some embodiments, the housing can be reused. In some embodiments, the housing can be reversibly opened and closed. In some embodiments, the housing can include at attachment ledge.

In some embodiments, the housing holds a needle so the bevel is facing towards user's eye; bevel is away from patient's iris.

In some embodiments, no sample moves beyond the collection chamber.

A housing can include a hub, which can be next to a handle portion of a housing and can be a pyramidal shape, bullet, or other suitable shape. A hub can be between about 4 mm and about 50 mm (e.g., about 4, 5, 10, 15, 20, 25, 30, 35, 40, 45, or 50 mm) in length, between about 4 mm and about 50 mm (e.g., about 4, 5, 10, 15, 25, 30, 35, 40, 45, or 50 mm) thick at the widest point, and between about 1 and about 5 mm (e.g., about 1, 2, 3, 4, or 5 mm) thick at the narrowest point. A hub can be removable to access elements contained inside a housing of a device. A hub can contain a hub assembly, which can include e.g., a needle for ocular fluid collection, a second needle, a collection chamber, a vacuum conduit, a luer connection, a dolphin nose tip, or combinations thereof.

Device Dolphin Nose Tip

In some embodiments, a device can include a dolphin nose tip that is designed to provide increased user control by direct placement of the device on the ocular surface. A dolphin nose tip geometry, material, and configuration can prevent corneal abrasions and/or tissue damage. A dolphin nose tip can be used to indent the anterior chamber of the eye and cause increased fluid egression into the device for collection without damage to the eye. A dolphin nose tip can prevent or reduce hand fatigue during ocular fluid collection and help with visualization of ocular fluid collection. A dolphin nose tip can orient a needle direction and bevel direction for safety. A dolphin nose tip can also be referred to as a combination penetration depth and abrasion limiter. A dolphin nose tip can be integral to the housing or hub. A dolphin nose tip can be removably attached to a housing or a hub.

A dolphin nose tip can have a rounded geometry, a convex geometry, a geometry that resembles the profile of a dolphin nose, a rounded pyramidal shape, a geometry made of a series of concentric discs stacked atop one another with decreasing circumference, a vertical section of a torus geometry, a half sphere geometry, or any other suitable shape. A diameter of the dolphin nose tip can be between about 2.00 and about 4.00 mm (e.g., about 2.00, 2.25, 2.50, 2.75, 3.00, 3.25, 3.50, 3.47, or 4.00 mm). A geometry of a dolphin nose tip can have a small hole through which a needle can extend centered in the dolphin nose tip (resembling, for example, the nipple of a baby bottle). A needle for ocular fluid collection can extend through a dolphin nose tip. A dolphin nose tip can decrease damage to the eye, rest on the eye with no damage to the eye, can rest on the eye to apply pressure to the eye, indent the eye to create more pressure, or combinations thereof. Indenting the eye to create more pressure can decrease the amount of time it takes to withdraw a given amount of fluid (e.g., 0.03, 0.04, 0.05, 0.06, 0.07, 0.08, 0.09, 0.10, 0.11, 0.12, 0.14, 0.15, 0.16, 0.17, 0.18, 0.19, or 0.20 mL) by about 1, 2, 3, 4, 5, 6, 7, 8, 9, 11, 12, 13, 14, 15, or more seconds. A user can insert the needle of a device described herein into the eye and rest the dolphin nose tip on the eye for increased stability during fluid collection.

A vacuum system can be configured to collect ocular fluid in a time period of about 30, 29, 28, 27, 26, 25, 24, 23, 22, 21, 20, 19, 18, 17, 16, 15, 14, 13, 12, 11, 9, 8, 7, 6, 5 seconds, or less with a pressure differential between about 50 mmHg to 70 mmHg negative pressure (e.g., about 50, 55, 60, 65, or 70 mmHg). A pressure differential is the difference in pressure between a vacuum system and an intra ocular pressure (IOP) of the eye.

Device with Vacuum System with an Optional Second Needle

Devices for ocular fluid collection are provided herein having a housing comprising a handle and a hub and a first needle having a gauge of 27, 28, 29, 30, 31, 32, 33, or 34. A collection chamber having a proximal and distal end can be disposed within the housing and coupled at its proximal end to a distal end of first needle for fluid collection. The collection chamber can be within the hub or the handle portion of the housing. Optionally, the collection chamber can be partially within the hub and partially within the handle portion of the housing.

A device can comprise a vacuum system within the housing configured to pull the fluid through the needle and into the collection chamber. A vacuum system can comprise a vacuum chamber; a compression mechanism connected to the vacuum chamber; and a mechanism to prevent retrograde flow of ocular fluid. A self-sealing pierceable vacuum septum can be present between the collection chamber and the vacuum chamber, e.g., a the of the vacuum chamber closest to the collection chamber. The vacuum system can further comprise a vacuum conduit positioned at the distal end of the collection chamber and connected to a second needle and a compression mechanism configured to push or pull the vacuum chamber towards or away from the vacuum conduit. The second needle can pierce the vacuum septum and user-controlled movement of the compression mechanism can be used to modulate the flow of the vacuum system thereby controlling the rate fluid is drawn into the collection chamber.

A mechanism to prevent retrograde flow of ocular fluid can be present and can comprise a valve such as a pinch valve, a microfluidic valve, a ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof.

Tubing such as a metal tube or elastomeric tubing, a molded polymeric sealed channel, or a microfluidic system can connect the collection chamber to the vacuum system.

A first needle for fluid collection can comprise an internal portion between about 14 mm and about 26 mm in length disposed inside the housing and an external portion between about 4 mm and about 6 mm in length disposed outside the housing for insertion into an eye.

The distal end of the collection chamber can comprise a fluid separation membrane, such as a semipermeable membrane, configured to prevent collected fluid from exiting the collection chamber or contacting the vacuum chamber. The device can provide a penetration force or between about 0.19 N and about 0.45 N. The device can be configured to collect the ocular fluid in a time period of about 20 seconds or less (e.g., about 20, 18, 16, 14, 12, 10, 9, 8, 7, 6, 5, 4, 3, 2, 1 or fewer seconds) with a pressure differential between about 50 mmHg to 70 mmHg (e.g., about 50, 55, 60, 65, or 70 mmHg) negative pressure.

A device can further comprise a visual indicator disposed on an exterior surface of the housing, which indicates when the collection chamber is full. A collection chamber can be removable and can configured to fit into a nucleic acid amplification device point of care diagnostic, or other diagnostic device.

A device can further comprise a dolphin nosed tip coupled to the housing or hub and coupled to the first needle.

A first needle can comprise a proximal and distal end, the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle; a primary bevel length (L3) between about mm and about 1.30 mm; and a lumen length (L2) between about 0.5 mm and 0.9 mm.

In some embodiments, a device can comprise a first needle 102 for ocular fluid collection, a vacuum system 108 and a second needle 116 for piercing a vacuum septum 112 (e.g., a self-sealing pierceable vacuum septum) of a vacuum chamber 110 (see e.g., FIG. 4 and FIG. 2). A vacuum chamber 110 and a collection chamber 104 can be evacuated upon activation by the user (e.g., slider 128 or retention latch mechanism 120) to provide a pressure differential between the collection chamber 104 and the eye. The device can also include a housing 106, which can include a handle portion 130 that can contain a vacuum system 108 and a hub 123 that can contain a hub assembly (see e.g., FIG. 4 and FIG. 2). A vacuum system 108 can include a vacuum chamber 110, at least one spring 118 operably coupled to a distal end of a vacuum chamber 104, and, e.g., a retention latch mechanism 120 (see e.g., FIG. 2) or a slider 128 (see e.g., FIG. 4). A vacuum system can include a vacuum septum 112 (e.g., a self-sealing pierceable vacuum septum). A hub assembly can include, for example, an interior portion of a needle 154 extending through a dolphin nose tip 142 and connected to a collection chamber 104 disposed in a luer connection 138, the collection chamber 104 connected with a vacuum conduit and the second needle 116 extending from the distal end of the collection chamber (see e.g., FIG. 4 and FIG. 2). In an embodiment the second needle can comprise a depth limitation device (a disk or other safety stopper) 114 so that the second needle does not penetrate too far into the vacuum device. The depth limitation device (a disk or other safety stopper) 114 can also be used to stabilize the second needle in the device.

A collection chamber 104 can include a proximal end 156 and a distal end 158. A collection chamber 104 can also include a fluid separation membrane, such as a semipermeable membrane 140 at the distal end 158 of the collection chamber 104 (see e.g., FIG. 4 and FIG. 2).

In some embodiments, a hub assembly and/or a collection chamber 104 can be disposable. A housing 106 and/or a vacuum system 108 can be reusable.

In some embodiments, a vacuum chamber 110 can compress a spring 118 located at a distal end of the vacuum chamber 110 and a retention latch mechanism 120 can secure the vacuum chamber 110 in a compressed position (see e.g., FIG. 2). Then, a distal end 158 of a collection chamber 104 can be inserted into a hub 123 as part of a hub assembly. An assembled needle 102, hub assembly (including, e.g., a vacuum conduit) can be attached to a handle 130. Upon a user fully inserting a needle 102 into the eye, the user presses, slides or otherwise engages a retention latch mechanism 120, which decompresses a spring 118 and pushes a distal end of a vacuum chamber 104 towards a proximal end of a vacuum conduit, which then pierces a vacuum septum 112 (e.g., a self-sealing pierceable vacuum septum) thereby connecting the vacuum chamber 110 to the collection chamber 104, creating an artificial pressure differential along the fluid path (see e.g., FIG. 2).

In some embodiments, a vacuum chamber 110 can compress a spring 118 located at a distal end of the vacuum chamber 110 and a slider 128 on the handle 130 portion of the housing 106 can move the vacuum chamber 110 forward to be pierced by a second needle 116 extending from the proximal end 156 of a collection chamber 104 (see e.g., FIG. 4). A second needle 116 can operate as a vacuum conduit. The assembly of a device using a slider 128 can be similar to the assembly described above in reference to a retention latch mechanism 120, but the slider 128 can keep the vacuum chamber 110 in position atop a compressed spring 118 (see e.g., FIG. 4B). Upon a user fully inserting a needle 102 into the eye, the user slides the slider 128, which decompresses a spring 118 and pushes a distal end of a vacuum chamber 104 towards a proximal end of a vacuum conduit, which then pierces a vacuum septum 112 (e.g., a self-sealing pierceable vacuum septum) thereby connecting the vacuum chamber 110 to the collection chamber 104, creating an artificial pressure differential along the fluid path (see e.g., FIG. 4A). The device configuration shown in FIG. 4 can give the user more control over the timing of the collection of the fluid from the eye by allowing intermittent engagement of a vacuum chamber 110 with the fluid collection path (i.e., connecting to the collection chamber 104). The device can prevent the risks of contamination and vision-inhibiting air bubbles in the anterior chamber.

The collection chamber 104 can include a fluid separation membrane, such as a fluid separation membrane 140 at the distal end to prevent any collected fluid traveling beyond the collection chamber 104 and into the vacuum chamber 110. The hub 123 and collection chamber 104 can be made of clear or translucent injection molded plastic that can enable the user to observe the accumulation of fluid within the collection chamber 104. The observation of fluid collection may be enhanced by the inclusion of a buoyant object within the collection chamber whose visible position within the chamber changes as it fills with fluid (e.g., FIG. 12A) or an LED indicator FIG. 12B. A device can allow for one-handed use by the physician and vacuum assisted fluid extraction for quicker procedural time and reduced retrograde flow of fluid. A device with a spring compression mechanism can prevent the risks of contamination and vision-inhibiting air bubbles in the eye.

Devices with Three Bevel Geometry

Devices for ocular fluid collection with three bevel geometry are provided herein. A device can comprise a housing comprising a handle and a hub, and a needle having a proximal and distal end, the needle comprising: a gauge of 27, 28, 29, 30, 31, 32, 33, or 34; the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about and about 30° with respect to a transverse axis of the needle; a primary bevel length (L3) between about 0.90 mm and about 1.30 mm; and a lumen length (L2) between about 0.5 mm and 0.9 mm; a collection chamber within the housing and coupled to the distal end of the needle; and any suitable vacuum system within the housing configured to pull the ocular fluid through the needle and into the collection chamber. The needle can comprise an internal portion between about 14 mm and about 26 mm in length disposed inside the housing and an external portion between about 4 mm and about 6 mm in length disposed outside the housing. The needle can comprise a lubricant coating, a hydrophilic polymer coating, an acrylic hydrogel polymer coating, a medical grade silicone lubricant, a crosslinked silicone lubricant, a vapor deposited polymer, a parylene coating, polydimethylsiloxane liquid coating, a dispersion containing 50 percent active silicone mixed in aliphatic and isopropanol solvents, a low residual coating, or combinations thereof.

The needle can comprise a total length between about 18.2 mm and about 19.7 mm and a wall thickness of about 0.03 mm to about 0.07 mm. The devices can provide a penetration force between about 0.19 N and about 0.45 N.

The collection chamber can be removable and can configured to fit into a nucleic acid amplification device, point of care diagnostic, or other diagnostic device. The collection chamber can hold a volume of about 0.05 0.1, 0.15, 0.20, 0.25, 0.30, or 0.35 mL.

The devices can further comprise a dolphin nose tip coupled to the housing.

The devices can further comprise a tube, molded polymeric sealed channel, or microfluidic system connecting the collection chamber to the vacuum system and a valve configured to prevent retrograde flow of the ocular fluid on the proximal side and control the vacuum force on the distal side of the collection chamber. The valve can comprise a pinch valve, ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof.

The vacuum system can comprise a vacuum chamber, a decompression handle, a vacuum conduit, vacuum septum, or combinations thereof. The vacuum system can be configured to collect the ocular fluid in a time period of 20, 15, 10, 5 seconds or less with a pressure differential between about 50 mmHg to 70 mmHg (e.g., about 50, 55, 60, 65, or 70 mmHg) negative pressure.

The devices can further comprise a visual indicator disposed on an exterior surface of the housing, which when the collection chamber is full.

Devices Comprising Integral Air-Filled Bulb

A device for ocular fluid collection comprising an integral air-filled bulb is provided herein. A device can comprise a housing comprising a hub and a handle and a deformable polymeric bulb disposed within the handle or housing. Mechanical compression (deformation) of the bulb establishes a pressure differential disposed within a handle portion of the housing when the mechanical compression is released. A device can comprise a 27, 28, 29, 30, 31, 32, 33, or 34 gauge needle in fluid communication with the device comprising a deformable polymeric bulb. The deformable polymeric bulb can be a transfer pipette that can include a tip portion, a neck portion, and a bulb portion. A needle for fluid collection can be attached to the air-filled bulb device, for example, at the tip of a transfer pipette.

The needle can comprise an internal portion between about 14 mm and about 26 mm in length disposed inside the housing and an external portion between about 4 mm and about 6 mm in length disposed outside the housing for insertion into an eye. The needle can provide a penetration force between about 0.19 N and about N. The collection chamber can hold a volume of about 0.05, 0.1, 0.15, 0.20, 0.25, or 0.35 mL. Ocular fluid can be collected into a tip portion, a neck portion, or a bulb portion of the transfer pipette. The device can include a ratcheting system that comprises a user operable bulb compression lever (150), a cocking knob (146), and a lock-out pin (148). The device further comprises a retrograde-flow prevention system configured to prevent ocular fluid from being returned to the eye by incorporation of a self-activated lock pin (148) that prevents the user from compressing the bulb without re-cocking the device.

The device can further comprise a ratcheting system that comprises a user operable bulb compression lever (150), a cocking knob (146), and a lock-out pin (148), The device of can further comprise a retrograde-flow prevention system configured to prevent ocular fluid from being returned to the eye by incorporation of a self-activated lock pin (148) that prevents the user from compressing the bulb without re-cocking the device. An air-filled bulb device can further comprise a self-sealing pierceable vacuum septum on a proximal end, e.g., at a transfer pipette tip. A device can further comprise a dolphin nose tip coupled to the hub and/or the needle.

A needle can comprise a proximal and distal end, the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle; a primary bevel length (L3) between about mm and about 1.30 mm; and a lumen length (L2) between about 0.5 mm and 0.9 mm.

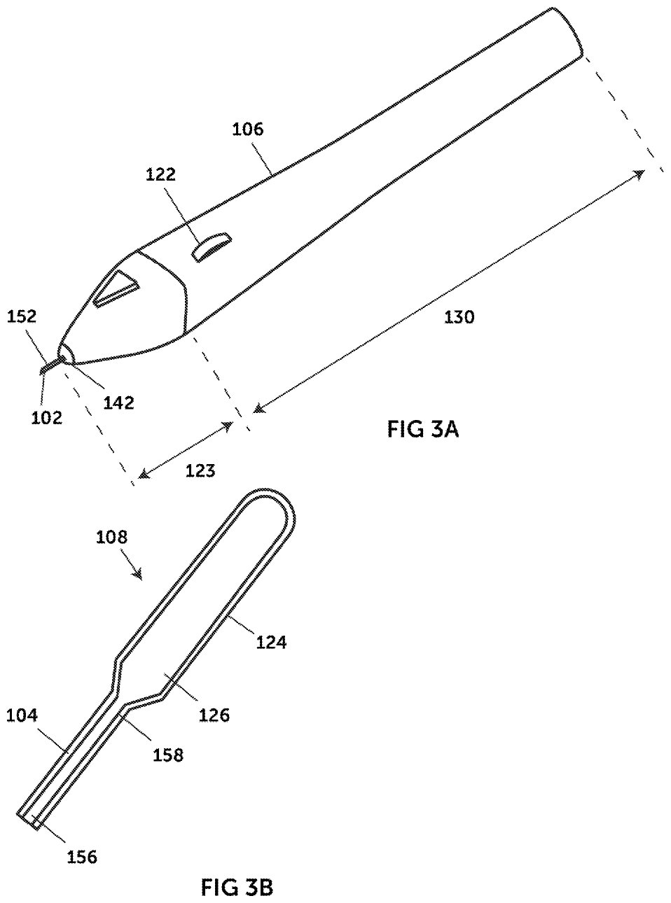

In some embodiments, the device can include a needle, and a housing 106 containing a vacuum system 108, which vacuum system 108 can be a displacement style vacuum system using a device with an integral air-filled bulb 126 such as a transfer pipette 124 as shown in both FIG. 3 and FIG. 5. In some embodiments, a transfer pipette 124 can provide a vacuum assist and include a collection chamber 104 and can be placed in a handle 130 portion of a housing 106 (see e.g., FIGS. 3 and 5). A transfer pipette 124 can include an integral air-filled bulb 126 and a fluid separation membrane, such as a fluid separation membrane at the proximal end of the transfer pipette 124. Prior to inserting the needle 102 into the eye, an air-filled bulb 126 can be compressed by the user pressing, sliding or otherwise engaging the mechanism that compresses the bulb (see e.g., FIGS. 3 and 5). The compression can be released after the eye has been punctured and the needle 102 is fully inserted into the eye, the negative pressure created by the expanding air-filled bulb 126 combined with the pressure within the eye can allow for quick fluid extraction into the collection chamber 104. Upon completion a transfer pipette 124 can be removed from a housing 106, properly packaged, and shipped to a lab for analysis by a nucleic acid amplification device (e.g., a PCR device), point of care, or other diagnostic procedure.

In some embodiments, a device can comprise a housing 106 with a handle 130 and hub 123 and a needle 102 for ocular fluid collection, as shown in FIG. 3. A hub 123 can contain an internal portion of the needle (not shown), a dolphin nose tip 142, a collection chamber 104, or combinations thereof. A handle 130 can include a vacuum system 108 which can include a transfer pipette 124 with an integral air-filled bulb 126. An integral air-filled bulb 126 can be deformably polymeric bulb and mechanical compression (deformation) of the integral air-filled bulb 126 can establish a pressure differential for ocular fluid collection into a collection chamber 104 of a transfer pipette 124 (see e.g., FIG. 3). In some embodiments, integral air-filled bulb 126 can be compressed by a user after a needle/hub assembly is secured, exhausting the volume of air inside integral air-filled bulb 126 out through the needle. In some embodiments, there can be a latching mechanism (not shown in FIG. 3) or a user can maintain pressure on a compression latch 122 until a needle 102 for ocular fluid collection is fully inserted into the cornea at which the compression latch 122 can be released and/or finger pressure on the compression latch 122 can be relaxed allowing the bulb to expand, creating negative pressure within a vacuum system 108 based on an integral air-filled bulb 126 (e.g., FIG. 3A). In some embodiments, a compression latch 122 can be used to modulate and/or throttle an amount of vacuum provided by a vacuum system 108. In some embodiments, a compression latch 122 could be used to compress an integral air-filled bulb 126 and another button (e.g., a release button, a disengagement button, a compression latch release, or other suitable element) elsewhere can release the compression latch 122.

As shown in FIGS. 5A-5D, in some embodiments, a device can include housing 106 with a handle 130 containing a ratcheting mechanism operably coupled to the transfer pipette 124 for ocular fluid collection. The device can also include a needle 102 for ocular fluid collection at, e.g. a tip of a transfer pipette. A ratcheting compression mechanism (i.e., ratcheting system) can include a vacuum chamber, a lever, a cocking knob, and a lock-out pin. A ratcheting compression mechanism can compress (deform) a deformable air-filled bulb of a transfer pipette to establish a pressure differential using a user operable bulb compression lever (150) that cannot be depressed until the cocking knob (146) is pulled outward from a handle, pulling with it a lock-out pin (148) (see e.g., FIG. 5). Air-filled bulb compression can be maintained by the user continuing to press on an exposed portion of a lever (150), the user slowly releases the lever (150) only after fully inserting a needle into the eye, when an air-filled bulb has fully decompressed a spring (not shown) pushes a lock pin (148) forward and under the end of the lever thereby preventing the user from compressing the bulb without re-cocking a device (see e.g., FIG. 5). A device can include a retrograde-flow prevention system configured to prevent ocular fluid from being returned to the eye by incorporation of a self-activated lock pin (148) that prevent the user from compressing the bulb without re-cocking the device (see e.g., FIG. 5). The device can require the use of multiple hands or an assistant to pull the ratchet component backwards to operate the ratcheting mechanism.

In some embodiments, a distal end of a needle for ocular fluid collection can pierce a vacuum septum located at a proximal end of a pipette, which can be placed in a handle; a hub 123 containing a suitable hub assembly can be attached to the handle of a device (e.g., with a push-twist motion) and can drive the distal end of the needle through the septum and secure the hub to the handle (not shown). In some embodiments, connecting the hub 123 to the handle 130 can incorporate a septum-less configuration having an airtight joint that would partially insert into or slide over the outside of the open end of a pipette.

Device with Decompression Handle

In some embodiments, a device can comprise a needle 102 for ocular fluid collection, a housing 106 with a hub 123 and a handle 130, and a decompression handle 132 configured to pull the fluid through the needle 102 and into a collection chamber 104 (see e.g., FIG. 6). A hub 123 can include an interior portion of a needle 154 extending through a dolphin nose tip 142, a collection chamber 104, tubing 134 (e.g., a metal tube, a molded polymeric sealed channel, elastomeric tubing, or a microfluidic system), a collection chamber septum 212 at a proximal end 156 of the collection chamber 104, and a fluid separation membrane, such as a semipermeable membrane 140 at a distal end 158 of the collection chamber 104 (see e.g., FIG. 6). A fluid separation membrane 140 can be configured to prevent collected fluid from contacting the vacuum chamber 110 and/or a luer connection 138. A collection chamber 104 can also be disposed in a luer connection 138 (see e.g., FIG. 6B). A handle 130 can include a vacuum system 108 (e.g., a decompression handle mechanism based vacuum system), which can include a vacuum chamber 110 operably coupled to a decompression handle 132 that can evacuate gas from the vacuum chamber 110 thereby establishing a vacuum state in the vacuum chamber 110. A vacuum system can also include a valve (e.g., a check valve or a microfluidics system, not shown) configured to prevent retrograde flow. A device with a decompression handle mechanism can further include a vacuum conduit to connect a vacuum system 108 to a collection chamber 104. A vacuum conduit can be, for example, elastomeric or other suitable tubing 134 or a microfluidic system disposed between a vacuum chamber 110 operably coupled to a decompression handle 132 and a collection chamber 104, which can be coupled to a needle 102 for ocular fluid collection (see e.g., FIG. 6). A tubing 134 (e.g., a metal tube, a molded polymeric sealed channel, or elastomeric tubing) can include a pinch valve 136 configured to modulate connection between a vacuum system 108 and a collection chamber attached to a needle 102 for ocular fluid collection (see e.g., FIG. 6A). A handle 130 can also include a pinch valve access button 137 for the user to engage a pinch valve 136 (see e.g., FIG. 6A and FIG. 7). In some embodiments, a hub assembly can be disposable, a collection chamber 104 can be disposable, a housing 106 can be reusable, a vacuum system 108 that can be reusable, or combinations thereof.

Initially, a user can pull back a decompression handle 132, which applies a vacuum force through an elastomeric 134 or other suitable tubing, a collection chamber 104, and a needle 102 to withdraw fluid from the eye. In some embodiments, a decompression handle 132 can be pulled back from the housing in preparation of using a device. A decompression handle 132 can be pulled out while using a device (e.g., during fluid collection). A decompression handle 132 can be configured such that when the decompression handle 132 is touching a housing 106 of a device, that can indicate the pressure in a vacuum chamber 110 no longer functions as a vacuum. A device can provide a penetration force between about 0.19 N and about 0.45 N. A collection chamber 104 can be removable and can be configured to fit directly into a nucleic acid amplification device, point of care diagnostic, or other diagnostic device. A device can also include an indicator (not shown) disposed on an exterior surface of the housing 106, which can indicate when the collection chamber 104 is full.

Methods

Also provided herein are methods for collecting ocular fluid. Methods can include inserting a needle of a device disclosed herein into an eye with minimal eye displacement, collecting ocular fluid through the needle, and removing the needle from the eye with reduced risk of injury. The methods and devices operate at a pressure that will not over deflate and damage the eye.

Methods for collecting ocular fluid, can comprise using any of the devices described herein to insert a needle into an eye with minimal eye displacement. The needle can be placed through the corneal tissue to access the anterior chamber of the eye. The vacuum system can be engaged so that ocular fluid flows through the needle into a collection chamber of a device. The vacuum can be released and the needle removed from the eye. Alternatively, the needle can be removed from the eye and then the vacuum is released. The device can notify the user with a signal from an indicator when a specific volume of fluid has been collected into a collection chamber of the device. A collection chamber can be removed from the device for use in nucleic acid amplification device, point of care diagnostic, or other diagnostic device.

Ocular fluid collection can be used to diagnose infection, cancer, and other diseases (e.g., dry eye, glaucoma, diabetes and neurodegenerative diseases). Ocular fluid collection is an emerging field with the use of ocular fluid as a diagnostic reservoir filled with proteins, immune cells, cytokines, and other signaling cells that can be used as biomarkers of ocular disease and systemic disease.

For example, some cancers can be found to alter the ocular fluid composition. Some infections have a predilection for ocular fluid (e.g., SARS-CoV-2). Inflammatory states can change throughout infection and can potentially be detected using ocular fluid biopsy.

The devices and collection methods described herein enable physicians to perform liquid biopsies of the ocular fluid. There is a true interest for monitoring levels of various protein markers or genetic markers in ocular fluid. Biomarkers that are tested from ocular fluid can include, e.g., VEGF, MMP-9, glycated albumin, LCN-1, lactotransferrin, Lysozyme C, lipophilin A, Ig Lamba chain, B2M, lacryoglobin, TNF-alpha, IgGa-Antichymotrypsin. Other eye health biomarkers can be expanded for ocular fluid diagnostics.

The compositions and methods are more particularly described below and the Examples set forth herein are intended as illustrative only, as numerous modifications and variations therein will be apparent to those skilled in the art. The terms used in the specification generally have their ordinary meanings in the art, within the context of the compositions and methods described herein, and in the specific context where each term is used. Some terms have been more specifically defined herein to provide additional guidance to the practitioner regarding the description of the compositions and methods.

As used herein, the term “and/or” includes any and all combinations of one or more of the associated listed items. As used in the description herein and throughout the claims that follow, the meaning of “a”, “an”, and “the” includes plural reference as well as the singular reference unless the context clearly dictates otherwise. The term “about” in association with a numerical value means that the value varies up or down by 5%. For example, for a value of about 100, means 95 to 105 (or any value between 95 and 105).

All patents, patent applications, and other scientific or technical writings referred to anywhere herein are incorporated by reference herein in their entirety. The embodiments illustratively described herein suitably can be practiced in the absence of any element or elements, limitation or limitations that are specifically or not specifically disclosed herein. Thus, for example, in each instance herein any of the terms “comprising,” “consisting essentially of,” and “consisting of” can be replaced with either of the other two terms, while retaining their ordinary meanings. The terms and expressions which have been employed are used as terms of description and not of limitation, and there is no intention that in the use of such terms and expressions of excluding any equivalents of the features shown and described or portions thereof, but it is recognized that various modifications are possible within the scope of the claims. Thus, it should be understood that although the present methods and compositions have been specifically disclosed by embodiments and optional features, modifications and variations of the concepts herein disclosed can be resorted to by those skilled in the art, and that such modifications and variations are considered to be within the scope of the compositions and methods as defined by the description and the appended claims.

Any single term, single element, single phrase, group of terms, group of phrases, or group of elements described herein can each be specifically excluded from the claims.

Whenever a range is given in the specification, for example, a temperature range, a time range, a composition, or concentration range, all intermediate ranges and subranges, as well as all individual values included in the ranges given are intended to be included in the disclosure. It will be understood that any subranges or individual values in a range or subrange that are included in the description herein can be excluded from the aspects herein. It will be understood that any elements or steps that are included in the description herein can be excluded from the claimed compositions or methods.

In addition, where features or aspects of the compositions and methods are described in terms of Markush groups or other grouping of alternatives, those skilled in the art will recognize that the compositions and methods are also thereby described in terms of any individual member or subgroup of members of the Markush group or other group.

The following are provided for exemplification purposes only and are not intended to limit the scope of the embodiments described in broad terms above.

### EXAMPLES

### Example 1: Optimization of Needle Characteristics in Order to Minimize Corneal Penetration Force

Optimization of needle characteristics were tested using Table 1 as a guide. Critical characteristics were identified and used to customize a needle for force testing.

TABLE 1
Weighting Assigned to Needle Characteristics vs Entrance Force, Slide Force

| | | | | | | | Anterior | Posterior |
|---|---|---|---|---|---|---|---|---|
| | | | Outer | Inner | Wall | Bevel | Bevel | Bevel |
| | Length | Gauge | Diam | Diam | Thick | Length | Angle | Angle |
| Entrance Force | 0.02 | 0.1 | 0.08 | 0.07 | 0.07 | 0.22 | 0.3 | 0.14 |
| Slide Force | 0.03 | 0.32 | 0.3 | 0.07 | 0.08 | 0.07 | 0.07 | 0.06 |
| Exit Force | 0.04 | 0.12 | 0.1 | 0.01 | 0.09 | 0.23 | 0.06 | 0.35 |

To Investigate the Geometry, Penetration Force and Cutting Profile of a Novel, Disposable Needle Tip for Aqueous Paracentesis.

In vitro testing was performed to assess the entrance force required with the novel needle tips. Ten 30-gauge needles (Standard of Care) were compared to ten novel commercial needles on a synthetic test medium (0.38 mm thick polyurethane film) and explanted bovine or porcine eyes. This synthetic material is used by ophthalmic original equipment manufacturers because of its uniform physical characteristics and superior signal-to-noise properties. Bovine or porcine eyes were harvested fresh. The majority of tests were performed using porcine eyes as they have similar corneal resistance compared to human eyes.

Definitions: Maximum Penetration Force=Maximum force by the insertion of the device during penetration. Joules=Area under the force displacement curve (maximum recorded value for porcine bovine model). Needle Slide/Glide=Area under the work curve after the full bore of the needle has penetrated the lower medium surface (or maximum value in porcine bovine model). Cutting Time=Time from the needle tip entering the medium until it exits the lower medium. Wedging=Time between when the bevel edge has cleared lower medium surface and cannula. A two-tailed, paired t-test will be used to analyze the difference in the means of maximum penetration forces and work functions for the two sets of needles. Expected results will be to achieve a 20% reduction in the amount of force and a 10% reduction in the amount of work needed for penetration.

A Needle Gauge Comparison Test

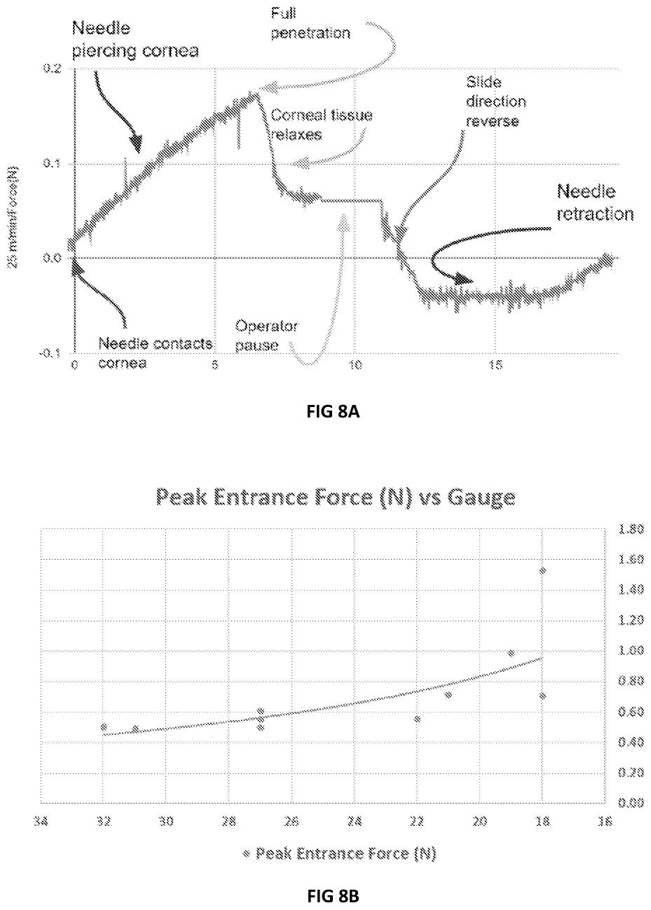

Investigation of the influence of needle gauge, or outside diameter, on the insertion force into the corneal. Several needles, with gauges ranging from 18G to 32G, were tested. Needles were lowered using a motorized test stand (Chatillon TCM-100) through a polyurethane film of thickness 0.015″ to a constant depth below the film. ForceTest software by Chatillon was used to display the force experienced by the needle as a function of time. This force is equal and opposite in direction to the force experienced by the film. The time series of this force includes several phases (FIG. 8A). The first phase occurs while the needle is in contact with the film but has yet to pierce the film. At the end of this phase is, at the moment of penetration, typically, the largest force of the insertion event. The second phase begins as force decreases after the needle penetrates the film and continues as the needle slides through the film. The third phase occurs as the needle reverses direction and retreats, still sliding through the film. The speed of the needle was 200 mm/min. which was controlled by the TCM-100. Although the sliding forces were taken into consideration, the maximum value from the force time series, termed the peak entrance force, was taken to be the most important. This decision was based on surgeon input that eye roll was due to act of entrance and not the resistance of the sliding force as the needle is further inserted. FIG. 8B displays an inverse relationship between needle gauge and peak entrance force, with best fit curve to help guide the reader. This trend was important in understanding the effect of gauge on penetrative forces for our novel needle.

Force Data Analysis

The ForceTest software produces force vs. time plots for the penetration tests. For much of the testing and analysis, a peak penetration force was used, which is that force which has the potential to cause the most damage to the patient and procedure. Table 2 shows how forces were determined from these plots

A Film-to-Eye Gauge Correlation Study

An inverse relationship between needle gauge and maximum entrance force was shown with polyurethane film, but further testing was needed in order to verify the results with the tissue of interest—corneal tissue. Using the same experimental layout, the film was replaced by bovine eyes (Animal Technologies, Tyler, TX), which shares similar properties to human corneal tissue (though it is slightly thicker than that of human eyes). The bovine eyes were penetrated from above the cornea, a top entry rather than a side entry. Although in an anterior chamber paracentesis procedure the physician would enter the cornea from the side, top entry enabled repeatable facilitation of experimentation. Eyes were held in place and partially pressurized them using a Vacuum Holder 4 Pigs (eyeCRE.AT GmbH, Austria), functioning as vacuum suction from beneath the eye. Further pressurization was achieved using an automotive hose clamp around the equator of the bovine globe. Eyes were pressurized to 18 mmHg, to simulate a healthy human eye pressure, and measured using a Schiotz Tonometer, as a control for pressure. The test stand was set to depress the needle until fully penetrated through the tissue, then after a short slide, retract the needle. Eyes were penetrated using four different types of needles with various gauges: 25G, 27G, 31G, and 34G. For each, three trials were performed where the associated peak entrance force was measured. The overall results show a correlation between higher needle gauge and lower entrance forces. More detailed inspection of the results, however, showed there was not a statistically significant difference between 31G and 34G needles (Table 2).

TABLE 2

| | Peak Value (N) | | |
|---|---|---|---|
| Needle Gauge | 1 | 2 | 3 |
| 25 G | 0.43 | 0.51 | 0.51 |
| 27 G | 0.30 | 0.33 | 0.36 |
| 31 G | 0.24 | 0.18 | 0.28 |
| 34 G | 0.23 | 0.26 | 0.18 |

This experiment generally verified that a higher gauge needle would require less insertion force in corneal tissue.

Primary Angle Variability

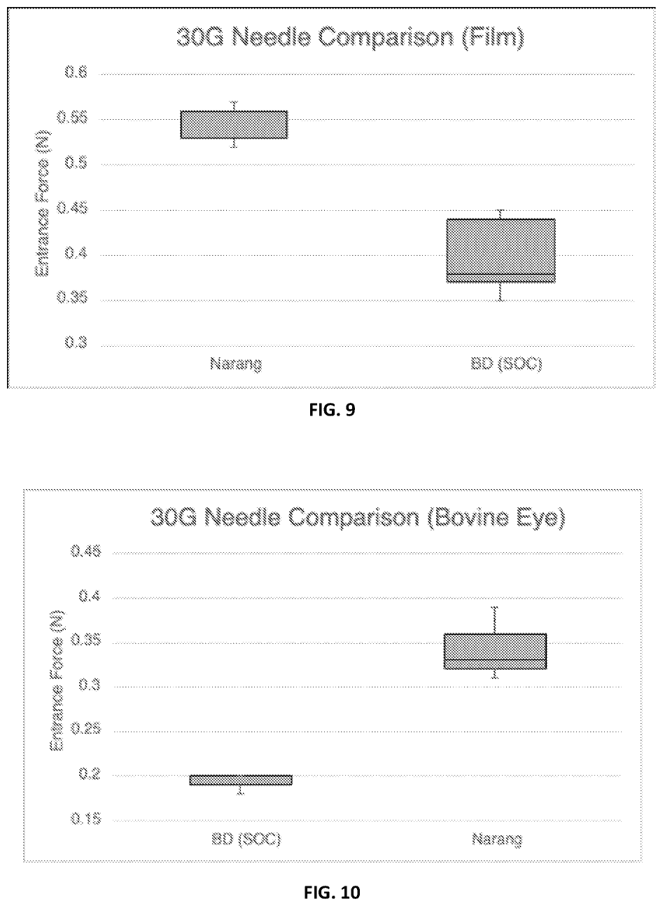

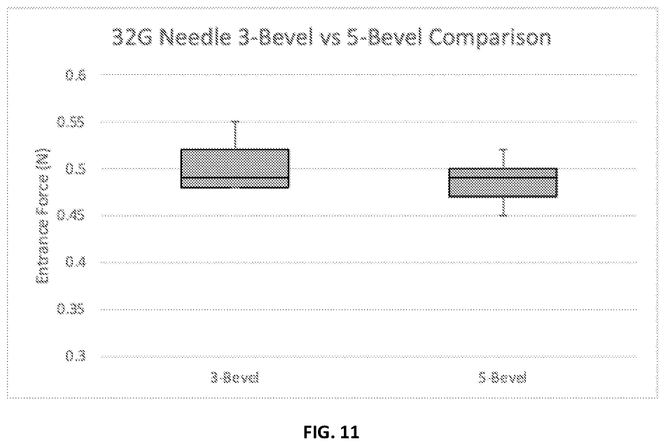

The purpose of this experiment was to determine the effect of primary angle on maximum entrance force. Two different types of 30G needles were obtained. Both had 3-bevel lancet geometry. The penetrated medium was 0.015-inch polyurethane film, and the penetration speed was 50 mm/min. The first needle was a disposable hypodermic needle produced by Narang Medical Limited made of 304 stainless steel and is ½″ long. It has a 3-bevel geometry, and the bevel dimensions are measured using Celestron Digital Microscope in combination with a calibrated quantitative measurement software. It has a primary bevel angle of 12.154°, a secondary bevel angle of 22.55°, and a bevel length of 1.272 mm. This needle will be referred to as the Narang needle. The second needle is a 30-gauge PrecisionGlide™ needle produced by BD, and it is the current standard of care (SOC). It is made of medical-grade 316 stainless steel and is 13 mm long. It also has a 3-bevel geometry, and its dimensions are measured by digital microscope. It has a primary bevel angle of 9.678°, a secondary bevel angle of 22.631°, and a bevel length of 1.399 mm. This needle will be referred to as the BD needle. Both needles have similar characteristics aside from their different primary angles. Each needle was tested five times each on the film. Recording force readings using the ForceTest software, the mean penetration force of Narang needles was found to be 0.548 N. The mean penetration force of BD needles was found to be 0.398 N (FIG. 9). A t-test shows statistical significance. The test result indicates that the BD needles require less penetration force to puncture through the film. Because nearly all characteristics of the needles were identical except for primary bevel angle, the measured difference in penetrative forces was a result of their difference in primary angle. This experiment helped to understand the relationship between primary bevel angle and penetration forces for the device.

A similar test was performed to determine the nature of this trend across mediums. An experiment was set up using bovine eyes rather than polyurethane film. This more closely matches the medium of this device's intended use. A similar trend in primary angle as it relates to penetrative force would exist was hypothesized. Largely, the same set-up was used for this experiment; however, rather than a thin polyurethane film being penetrated, bovine corneal tissue were penetrated. The same BD and Narang needles were tested using the same pressure and set-up. Eyes were pressurized and secured using the Vacuum Holder 4 Pigs with the addition of an automotive hose clamp and the force stand was lowered at 50 mm/min. After running the experiment, the penetration force of Narang needles was found to have a mean of 0.343 N and the penetration force of BD needles had a mean of 0.193 N (FIG. 10). This difference was found to be statistically significant with a t-test. Similar to the results of the film-based test, the BD PrecisionGlide needles, those with a lower primary angle, had significantly lower entrance forces through corneal tissue. Since nearly all other factors were held constant, differences in entrance force were attributed to differences in primary angle. Hence, these studies have shown that a lower primary angle, all things held constant, results in lower peak needle penetration force. This experiment reinforced the importance of a low primary angle on the needle design.

A Number of Bevels Comparison to Determine its Effects

The purpose of this experiment was to determine the impact of the number of bevels on a lancet needle as it relates to penetrative force. The number of bevels in the lancet bevel geometry can affect peak needle penetrative forces. Past studies of this comparison have involved needles of a much lower gauge and have not been tested on corneal tissue. The number of faces on a high-gauge lancet bevel influences the peak penetrative forces was investigated. Two different 32-gauge needles manufactured by BD were used. One needle had three bevels and a primary bevel angle of 8.32°, and a secondary bevel angle of 19.68°. The five-bevel needle has a primary angle of 9.563°, and a secondary bevel angle of 17.191°. The slight differences in bevel angles were treated as obsolete for the purposes of this experiment. Using the Chatillon TCM-100 test stand and the standard film testing setup with 0.015-inch film, each needle for 5 trials was tested. After running trials, the penetration force of the 3-beveled needles has a mean of 0.504 N whereas the penetration force of the 5-beveled needles has a mean of 0.486 (FIG. 10). This was found not to be a statistically significant difference. This experiment was important in determining a needle geometry for device design.

An Investigation of Dolphin Nose Tip-Induced Corneal Abrasions

The dolphin nose tip (see e.g., FIGS. 2A, 3A, 4, 6, 7, and 12) is a type of hub which is the interface that connects a cannula (i.e., needle) to the rest of the device. Hub design can minimize fluid loss, prevent corneal scratching, and enables improved positioning of the device to varied patient facial features. Corneal abrasions, though self-healing, can be painful and impair vision for a patient. The purpose of this experiment was to better understand how the shape of the hub tip can influence corneal impacts and abrasions. As common practice in human and veterinarian ophthalmology, viewing corneal abrasions is done with fluorescein dye. Traditionally, the dye is placed in the eye, followed by several patient blinks to fully cover the cornea. UV light is then used to view the abrasion. A similar procedure was performed to determine scratches caused by hub contact with the cornea. Three needle hub shapes were selected to test for optimal dolphin nose tip geometry, where two of the hub had flat head ends with distinct edges, the other hub had a convex hub geometry (i.e., a dolphin nose top as a combination penetration depth and abrasion limiter). All needle hubs were made of polypropylene, to isolate differences in material. With each needle, for three trials each, porcine eyes near the limbus from the side were entered. Needles were penetrated deep enough for the hub to touch the cornea. The operator touched the hub to the cornea and released three times followed by two twists with the hub against the cornea. Intentionally attempting corneal damage was done to mimic poor physician procedure to understand the risk of damage in the event of a mistake. The findings showed differences between hub geometries as it relates to magnitude of corneal abrasions. The results show that hub geometry can have a notable impact on corneal abrasion magnitude. This experiment was important because it provided information on dolphin nose tip design for minimization of corneal abrasions. The findings specifically support a combination penetration depth and abrasion limiter type of dolphin nose tip such that the hub-needle intersection point has convex curvature to reduce edge-related damage.

### Example 2: Creation of Novel Microfluidic Chamber with Check Valve

Design and validate Tesla check valve.

The use of a check valve in the design will allow for two flow characterizations: (1) laminar when dovetailed with the demands of the system, and (2) turbulent when retrograde. Given the pressure differential, this will stop the flow from reversing and keep it laminar. This modelling was done with the Hagen-Poiseuille equation detailed below.

| Hagen-Poiseuille Model | Q = (πPr^{∧}4)/(8ηl) | |
|---|---|---|
| Q | Flow (cm^{∧}3/second) | 0.001308892 |
| π | π | 3.141592654 |
| P | Pressure differential | 1999.84 |
| | (Pascal) | |
| r | Radius of the channel (cm) | 0.05 |
| η | Healthy Viscosity (Pa · s) | 0.75 |
| I | Length of the channel (cm) | 5 |

A Reynolds Number threshold of 2100 will indicate when flow has converted from laminar to turbulent. LINDO optimization software will be used, inputting necessary constraints such as depth of channel (mm), needle length (2.2 mm), gauge of needle (27), bevel length (1 mm), and non-needle length (1 mm). Flow testing and analysis will be conducted using a Model OV7670 CMOS camera, bovine eye fluid (obtained from needle penetration studies), colored dye, and transparent PDMS walls. This way, the flow of ocular fluid through the system of tesla valves can be assessed for its efficacy in preventing retrograde flow and facilitating laminar flow through the device into the collection chamber.

TABLE 3
Needle and Collection Device Modeling Parameters

| | |
|---|---|
| Chamber Volume | 250 mL |
| Corneal Wall Thickness | 1 mm |
| Density | 1000 kg m^{∧-}3 |
| Diameter | 0.1 mm |
| Needle Gauge | 22, 30, 32 gauge |
| Laminar Flow Threshold | 2100 |
| | Reynolds |
| | Number |
| Length of | 50mm |
| Microfluidic | |
| Channel | |
| Length of Needle | 0.5 in |
| Pressure in Human Eye | 1999.84 Pa |
| Pressure in Device | 0 Pa |
| Pressure Differential | 1999.84 Pa |
| Production Rate per Minute | 0.2 mL |

TABLE 4
Needle and Collection Device Modeling Parameters

| | | |
|---|---|---|
| Chamber Volume | 25 | cL |
| | 250 | mL |
| | 250000 | μL |
| Corneal Production Rate | 0.2 | cL |
| | 2 | mL |
| | 2000 | μL |
| Corneal Wall Thickness | 0.1 | cm |
| | 1 | mm |
| | 1000 | μm |
| Density | 1000 | kg m^{∧}−3 |
| Diameter | 0.01 | cm |
| | 0.1 | mm |
| | 100 | μm |
| Needle Gauge | 22 | Gauge |
| | 30 | Gauge |
| | 32 | Gauge |
| Laminar Flow Threshold | 2040 | Rn |
| Length of | 5 | cm |
| Microfluidic | | |
| Channel | | |
| | 50 | mm |
| | 50000 | μm |
| Length of Needle | 0.5 | in |
| Length of Short Needle | | |
| Pressure in Human Eye | 1999.84 | Pa |
| Pressure in Device | 0 | Pa |
| Pressure Differential | 1999.84 | Pa |
| Production Rate per Minute | 0.2 | cL |
| | 2 | mL |
| | 2000 | μL |
| Radius | 0.005 | cm |
| | 0.05 | mm |
| | 50 | μm |
| Turbid Flow Threshold | 2040 | Reynolds |
| | | Number |
| Viscosity | | |
| Healthy | 0.75 | Pa · s |

Designing a Collection Chamber with Vacuum-Sealed Compartments and PCR-Compatible Dimensions and Material Properties.

### Example 2.3: To Investigate Fluid Flow Rates in Non-Vacuum-Assisted Simulation

The purpose of this example was to determine fluid flow rates through various needle gauges to better understand how needle gauge affects fluid flow. The goal is to reduce collection time to approximately 20 seconds or less to avoid patient discomfort and reduce injury risk. A pressure-based simulation was developed to mimic the pressure gradient present in a non-vacuum assisted biopsy device.

In order to simulate the pressure differential between atmospheric pressure and intraocular pressure, a pressure differential was created over the length of the needle. In the experimental setup, the outlet of the needle was to be kept at atmospheric pressure, and, therefore, the inlet of the needle would need to be at a positive pressure of 18 mmHg to mimic that of a healthy human eye. This positive pressure was created with the use of an “L” shaped tubing filled with water, with graduations on the vertical tubing. The needle punctured the end of the horizontal section of the “L” shape through a water-tight rubber material. Water was used for experimentation because the kinematic viscosity is nearly identical to aqueous humor fluid. This height of the water was determined using the formula ΔPP=ρρρρh, where ΔPP is the difference in pressure between the vertical distance from the needle entrance to the top of the water column, pp is the density of water, g is the gravitational constant, and h is the vertical distance as described. The height of the column was determined to be 24 cm. The time for the column of water to drop by 200 uL was taken as the time needed collect the goal amount of fluid under 18 mmHg pressure.

A comparison was run between two types of needles that varied in gauge. One needle was 30 gauge while the other was 33 gauge. Each was tested in the fixture for three trials to understand how needle gauge affects fluid flow rate out of a pressurized compartment. The average time for 200 uL of fluid to be released was 77 seconds for the 30-gauge needle and 234 seconds for the 33-gauge needle. This gave an indication that higher needle gauge results in slower fluid extraction.

This experiment setup replicates the fluid dynamics of removing ocular fluid from the eye unassisted by vacuum. Not only does it replicate the pressure differential from the eye to the collection device, but it also replicates the experimental pressure differential decrease as the fluid height in the vertical column decreases due to the fluid flow through the needle just as the pressure decreases in the anterior chamber as fluid flows from the needle into the collection device.

Using the results, a resistance of the needle was calculated using an Ohm's law translation to fluid dynamics of P=RVFR where P is the pressure differential, R is the resistance due to the needle, and VFR is the volumetric flow rate of the fluid. Although the flow rate of the water changes as the pressure differential decreases in the experimental setup, the flow rate was assumed to be constant for rough calculations. Thus the 200 uL per 77 and 234 seconds yields a flow rate of approximately 2.6 and 0.85 uL/s, respectively. The desired flow rate is 200 uL in 20 seconds or 10 uL/s. Using P=RVFR and assuming the resistance is constant for different flow rates, roughly calculating, it can be seen that pressure differential of approximately 1, 2, 3, 4, 5, 6, 7, or 8 times 18 mmHg or 90 mmHg would roughly achieve the desired collection of 200 uL in 20 seconds.

Determining Vacuum Impact on Collection Parameters

Simulations were performed for a 30-gauge needle. Unlike the physical experiments that were performed, the simulations assumed a constant pressure within the eye. As described earlier, the pressure within the eye will decrease as fluid is removed at the rate desired. Thus, the simulations are a “best-case” scenario. The pressure that produced the desired collection time from the simulations would necessarily need to be higher in application. Nevertheless, this simulation gave a good benchmark to compare initial experimental results. The simulation showed that a collection time of 20 seconds was achieved with an approximately 50 mmHg to 70 mmHg negative pressure differential (considering the approximate 18 mmHg eye pressure). Since human intraocular pressure should be between 10 and 21, collection of 200 uL in about 20 seconds or fewer was determined not to be possible without the use of an artificial pressure differential using vacuum.

All publications and patent applications mentioned in this specification are herein incorporated by reference to the same extent as if each individual publication or patent application was specifically and individually indicated to be incorporated by reference.

While the invention has been described in connection with various embodiments, it will be understood that the invention is capable of further modifications. This application is intended to cover any variations, uses or adaptations of the invention following, in general, the principles of the invention, and including such departures from the present disclosure as, within the known and customary practice within the art to which the invention pertains.

We claim:

1. A device for ocular fluid collection, the device comprising:
a housing comprising a handle and a hub;
a first needle having a gauge of 27, 28, 29, 30, 31, 32, 33, or 34;
a collection chamber having a proximal and distal end disposed within the housing and coupled at the proximal end to a distal end of the needle; and
a vacuum system within the housing configured to pull the ocular fluid through the needle and into the collection chamber, the vacuum system comprising:
a vacuum chamber; and
a compression mechanism connected to the vacuum chamber; and a mechanism to prevent retrograde flow of ocular fluid.

2. The device of claim 1, wherein tubing, a microfluidic system, or a molded polymeric sealed channel connects the collection chamber to the vacuum system.

3. The device of claim 1, further comprising self-sealing pierceable vacuum septum between the collection chamber and the vacuum chamber.

4. The device of claim 1, wherein the needle comprises an internal portion between about 14 mm and about 26 mm in length disposed inside the housing and an external portion between about 4 mm and about 6 mm in length disposed outside the housing for insertion into an eye.

5. The device of claim 1, further comprising a mechanism to prevent retrograde flow of ocular fluid comprising a pinch valve, a microfluidic valve, a ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof.

6. The device of claim 3, wherein the vacuum system further comprises:
a vacuum conduit positioned at the distal end of the collection chamber; and a compression mechanism configured to push or pull the vacuum chamber towards or away from the vacuum conduit.

7. The device of claim 1, wherein the vacuum conduit is a second needle.

8. The device of claim 1, wherein the distal end of the collection chamber comprises a fluid separation membrane configured to prevent collected fluid from exiting the collection chamber or contacting the vacuum chamber.

9. The device of claim 1, wherein the device provides a penetration force between about 0.19 N and about 0.45 N.

10. The device of claim 1, wherein the collection chamber is removable and is configured to fit into a nucleic acid amplification device, point of care diagnostic, or other diagnostic device.

11. The device of claim 1, wherein the device is configured to collect the ocular fluid in a time period of about 20, 15, 10, 5 seconds or less with a pressure differential between about 50 mmHg to 70 mmHg negative pressure.

12. The device of claim 1, further comprising a visual indicator disposed on an exterior surface of the housing, which indicates when the collection chamber is full.

13. The device of claim 1, further comprising a dolphin nose tip coupled to the housing.

14. The device of claim 1, wherein the first needle comprises:
a proximal and distal end, the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle;
a primary bevel length (L3) between about 0.90 mm and about 1.30 mm; and
a lumen length (L2) between about 0.5 mm and 0.9 mm.

15. A device for ocular fluid collection, the device comprising:
a housing comprising a handle and a hub;
a needle having a proximal and distal end, the needle comprising:
a gauge of 27, 28, 29, 30, 31, 32, 33, or 34;
the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about with respect to a transverse axis of the needle,
a primary bevel length (L3) between about 0.90 mm and about 1.30 mm, and
a lumen length (L2) between about 0.5 mm and 0.9 mm;
a collection chamber within the housing and coupled to the distal end of the needle; and
a vacuum system within the housing configured to pull the ocular fluid through the needle and into the collection chamber.

16. The device of claim 15, wherein the needle comprises an internal portion between about 14 mm and about 26 mm in length disposed inside the housing and an external portion between about 4 mm and about 6 mm in length disposed outside the housing.

17. The device of claim 15, wherein the needle comprises a lubricant coating, a hydrophilic polymer coating, an acrylic hydrogel polymer coating, a medical grade silicone lubricant, a crosslinked silicone lubricant, a vapor deposited polymer, a parylene coating, polydimethylsiloxane liquid coating, a dispersion containing 50 percent active silicone mixed in aliphatic and isopropanol solvents, a low residual coating, or combinations thereof.

18. The device of claim 15, wherein the device provides a penetration force between about 0.19 N and about 0.45 N.

19. The device of claim 15, wherein the needle comprises a total length between about 18.2 mm and about 19.7 mm and a wall thickness of about 0.03 mm to about 0.07 mm.

20. The device of claim 15, wherein the collection chamber is removable and is configured to fit into a nucleic acid amplification device, point of care diagnostic, or other diagnostic device.

21. The device of claim 15, wherein the collection chamber holds a volume of about 0.1, 0.15, 0.20, 0.25, 0.30, or 0.35 mL.

22. The device of claim 15, wherein the device further comprises a dolphin nose tip coupled to the housing.

23. The device of claim 15, wherein the device further comprises a tube, microfluidic system, or molded polymeric sealed channel connecting the collection chamber to the vacuum system.

24. The device of claim 23, wherein the device further comprises a valve configured to prevent retrograde flow of the ocular fluid comprising a pinch valve, ball check valve, a diaphragm check valve, a swing check valve, a flapper valve, a stop-check valve, a lift-check valve, an in-line check valve, a duckbill valve, a pneumatic non-return valve, a Tesla check valve, or combinations thereof.

25. The device of claim 15, wherein the vacuum system comprises a vacuum chamber, a decompression handle, a vacuum conduit, vacuum septum, or combinations thereof.

26. The device of claim 15, wherein the vacuum system is configured to collect the ocular fluid in a time period of about 20, 15, 10, 5 seconds or less with a pressure differential between about 50 mmHg to 70 mmHg negative pressure.

27. The device of claim 15, further comprising a visual indicator disposed on an exterior surface of the housing, which indicates when the collection chamber is full.

28. A device for ocular fluid collection, the device comprising:
a housing comprising a hub and a handle;
a device comprising a deformable polymeric bulb configured such that mechanical compression of the bulb establishes a pressure differential disposed within a handle portion of the housing when the mechanical compression is released; and
a 27, 28, 29, 30, 31, 32, 33, or 34 gauge needle in fluid communication with the a device comprising a deformable polymeric bulb.

29. The device of claim 28, wherein the deformable polymeric bulb is a transfer pipette comprising a tip portion, a neck portion, and a bulb portion.

30. The device of claim 28, wherein the needle provides a penetration force between about 0.19 N and about 0.45 N.

31. The device of claim 29, wherein the ocular fluid is collected into a tip portion, a neck portion, or a bulb portion of the transfer pipette.

32. The device of claim 28, wherein the device further comprises a ratcheting system that comprises a user operable bulb compression lever (150), a cocking knob (146), and a lock-out pin (148).

33. The device of claim 28, wherein the device further comprises a retrograde-flow prevention system configured to prevent ocular fluid from being returned to the eye by incorporation of a self-activated lock pin (148) that prevents the user from compressing the bulb without re-cocking the device.

34. The device of claim 28, wherein the device further comprises a dolphin nose tip coupled to the hub.

35. The device of claim 28, wherein the needle comprises:
a proximal and distal end, the proximal end comprising a three bevel geometry having a primary bevel surface 111 having a primary bevel angle (Ap) between about 5° and about 10° with respect to a longitudinal axis of the needle, a secondary bevel surface 113 having an angle (As) between about 9° and about 18° with respect to a longitudinal axis of the needle, and a tertiary bevel surface 115 having an angle (As) between about 9° and about 18° with respect to a longitudinal axis of the needle, the secondary bevel surface and the tertiary bevel surface having a tertiary bevel angle (At) between about 15° and about 30° with respect to a transverse axis of the needle;
a primary bevel length (L3) between about 0.90 mm and about 1.30 mm; and
a lumen length (L2) between about 0.5 mm and 0.9 mm.

36. A method for collecting ocular fluid, the method comprising:
using the device of claim 1, 15, or 28, inserting a needle into an eye with minimal eye displacement;
collecting ocular fluid through the needle into a collection chamber; and
removing the needle from the eye.

37. The method of claim 36, further comprising notifying the user with a signal from an indicator when fluid has been collected into a collection chamber of the device.

38. The method of claim 36, further comprising removing the collection chamber from the device for use in nucleic acid amplification device, point of care diagnostic, or other diagnostic device.
